(19) United States
(12) Patent Application Publication
(10) Pub. No.: US 2019/0160207 A1
(43) Pub. Date: May 30, 2019

(54) IMPLANT SURFACES FOR PAIN CONTROL

(71) Applicant: THE GENERAL HOSPITAL CORPORATION, Boston, MA (US)

(72) Inventors: Jeremy V. SUHARDI, Boston, MA (US); Ebru ORAL, Newton, MA (US); Orhun K. MURATOGLU, Cambridge, MA (US); Hany BEDAIR, Wellesley, MA (US)

(73) Assignee: THE GENERAL HOSPITAL CORPORATION, Boston, MA (US)

(21) Appl. No.: 16/098,233

(22) PCT Filed: Apr. 27, 2017
(86) PCT No.: PCT/US2017/029789

§ 371 Date: Nov. 1, 2018

Related U.S. Application Data
(60) Provisional application No. 62/330,478, filed on May 2, 2016.

(51) Int. Cl. A61L 27/54; A61L 27/48; A61K 31/451; A61K 31/167

(52) U.S. Cl. CPC A61L 27/54; A61L 27/48; A61K 31/451; A61L 2300/602; A61L 2300/402; A61L 2300/428; A61K 31/167

(57) ABSTRACT

The invention related to therapeutic polymeric materials and medical implants containing additives and/or analgesic agents. The invention also relates to methods of making therapeutic polymeric materials and medical implants containing additives and/or analgesic agents. Methods of spatially controlling additive concentrations and release as well as polymeric material morphology are also provided.

This application claims priority to U.S. Provisional Application No. 62/330,478 (both versions), filed May 2, 2016, the contents of which is hereby incorporated by reference into this application.

### FIELD OF THE INVENTION

The present invention relates to methods of making therapeutic polymeric materials and medical implants containing such materials. Methods of making medical implants containing additives and polymers and materials used therewith also are provided. Methods of spatially controlling additive concentrations and release as well as polymeric material morphology are also provided.

### BACKGROUND OF THE INVENTION

Joint replacement surgery has revolutionized the treatment of debilitating arthritis by reducing pain and restoring function of the joint. Many advancements have occurred in the technology and surgical technique of these procedures including more reliable fixation of implants, more favorable wear properties of the implants (and thus longevity), less surgical trauma and avoidance of early ambulation and directed physiotherapy. The one issue that has not significantly been improved upon is the control of post-operative surgical pain. These surgeries are extremely painful procedures that involve significant surgical dissection and osteotomies (cutting of the bone) to complete the joint replacement.

The current status of post-operative joint replacement pain management represents a slow evolution. Traditionally, patients were administered opiate-type pain medicine intravenously either by the care-giver or via a patient controlled analgesia device (PCA), but this has had many undesirable side effects including nausea, vomiting, confusion, delirium, and prolonged hospital stays. Recently, the trend has been towards the injection of a local anaesthetic into the surgical site at the time of surgery, providing the patient with immediate post-operative pain that can last several hours. This approach can be supplemented with regional anaesthetic nerve blocks, spinal anaesthesia, or epidural anaesthesia. While these techniques sometimes work very well, they are often unpredictable with results varying widely among patients. Even in the cases where these modalities work as planned, patients almost always require supplemental oral narcotics, particularly once the local anaesthetic has worn off (usually within the first 24 hours). This post-operative pain can result in poor functional outcomes of the procedure since patients find it difficult to participate in physiotherapy secondary to pain; this physiotherapy in the early post-operative period is critical to a favorable outcome. Patients frequently stay in the hospital several days until their pain is controlled and then discharged with these narcotics to help control post-operative pain that can last for months after surgery.

The problem that needs to be addressed is post-operative pain following joint replacement surgery. Even with the most current modalities of treatment, patients almost always require oral pain medicine, commonly in the form of narcotics. The pain following joint replacement surgery is a local problem where the pain generator and patient perception of pain is all localized to the surgical site.

The current treatment for this pain involves systemic drug administration to treat a local problem. This systemic administration often inadequately treats the pain. In order to achieve appropriate levels of pain control, patients often require medication doses that pose significant side-effects including delirium, nausea, vomiting, constipation, dizziness, ileus, and prolonged hospital stays. The ideal treatment would be an anaesthetic infusion into the surgical site at the time of perceived pain. This would treat the local pain generator and patient perception without the delivery of any systemic medication and its resultant side effects. Unfortunately, the only methods available to accomplish this are only temporary and short-lived and include repeated percutaneous injections of the drug directly into the joint or leaving a catheter in the joint that is connected to a drug infusion pump. The potential risk of this not only includes patient discomfort, but the increased risk of infection, a devastating complication that most physicians will avoid at all cost, making these options unviable.

A non-invasive method to deliver a local anaesthetic to the surgical site in a sustained manner for the first several weeks after joint replacement surgery promises to address all the problems outlined above. A clinically meaningful and sustained level of anaesthetic within the joint space will eliminate the need for oral and/or intravenous pain medicine and thus eliminate all side effects associated with those drugs. The pain relief should be immediate and sustained for several weeks after surgery. This would allow patients to be discharged from the hospital earlier. This would provide patients a pain-free experience, particularly during the critical physiotherapy sessions, facilitating and optimizing the gain from those sessions and reducing post-operative stiffness and pain. A local anaesthetic would not interfere with muscle function or strength, a common problem with regional anaesthetics and nerve blocks. Above all, this would reduce the morbidity associated with the otherwise highly successful surgical procedure of joint replacement surgery.

The solution to the problem of post-operative pain following joint replacement surgery is to deliver a local anaesthetic to the surgical site by means of the implanted polyethylene bearing used in almost all joint replacements. The polyethylene (a polymer) piece of the prosthetic device can be engineered to contain clinically relevant levels of an anaesthetic (AT) or a mixture of anaesthetics, such as lidocaine, bupivacaine, ropivacaine, or others embedded within the polymer. Once in vivo, the AT slowly and predictably elutes from the material, bathing the joint and local tissues with the anaesthetic, thus providing complete relief from pain after the procedure. The polyethylene polymer is manufactured and designed in such a way that AT elutes relatively quickly in the first few days after surgery, when the pain is most severe. In addition, the AT continues to elute from the material more slowly for a period of several weeks until most or all of the drug is eluted from the polymer, thus provide sustained pain relief until most tissues have healed.

### SUMMARY OF THE INVENTION

According to this invention, methods of incorporating therapeutics and specifically anesthetics and/or analgesics into a polymeric matrix are described. In some of the embodiments in this invention, the therapeutic(s) incorporated into a polymeric matrix is intended for release when in contact with water or bodily fluids. The incorporation into the polymer matric can be done in several ways. For example, anesthetics and/or analgesics, such as bupivacaine hydrochloride and/or ropivacaine hydrochloride powder, can be mechanically mixed with polyethylene powder prior to consolidation and then elute out when placed in water. Alternatively or in combination, anesthetics and/or analgesics, such as lidocaine, bupivacaine, and/or ropivacaine can be diffused into polyethylene at elevated temperatures and then elute out when placed in water at body temperature. Anesthetics and/or analgesics, such as bupivacaine hydrochloride or ropivacaine hydrochloride powder, mechanically mixed with polyethylene powder prior to consolidation and subsequently exposed to melted lidocaine, bupivacaine, and/or ropivacaine to allow diffusion of the aforementioned drugs into the polyethylene.

### Blending and Consolidation

In one embodiment, the invention provides a method of making a therapeutic, consolidated polymeric material comprising (a) providing a polymeric material; (b) blending the polymeric material with at least one therapeutic additive; and (c) consolidating the blend; thereby obtaining a therapeutic, consolidated polymeric material. Blending of the additive with the polymeric material can be direct mechanical mixing. Alternatively, the blending can be solvent-assisted by dissolving or dispersing the additive in a solvent and mixing the solution or dispersion with the polymeric material and then drying the solvent. The polymeric material can also be blended with other additives that are not incorporated for therapeutic purposes in addition to being blended with therapeutic additives.

In one embodiment, the invention provides a method of making an oxidation-resistant therapeutic polymeric material comprising (a) providing a polymeric material; (b) blending the polymeric material with an antioxidant; (c) blending the antioxidant-blended polymeric material with at least one therapeutic additive; and (d) consolidating the blend; thereby obtaining an oxidation-resistant therapeutic polymeric material.

In one embodiment, the invention provides a method of making a therapeutic polymeric material comprising (a) providing a polymeric material; (b) blending the polymeric material with lidocaine, bupivacaine, and/or ropivacaine; and (c) consolidating the blend; thereby obtaining a therapeutic polymeric material.

In one embodiment, the invention provides a method of making an oxidation-resistant therapeutic polymeric material comprising (a) providing a polymeric material; (b) blending the polymeric material with an antioxidant; (c) blending the antioxidant-blended polymeric material with lidocaine, bupivacaine, and/or ropivacaine; and (d) consolidating the blend; thereby obtaining an oxidation-resistant therapeutic polymeric material.

In one embodiment, the invention provides a method of making an analgesic polymeric material comprising (a) blending the polymeric material with lidocaine, bupivacaine, and/or ropivacaine; and (b) consolidating the blend; thereby obtaining an analgesic polymeric material.

In one embodiment, the invention provides a method of making a therapeutic medical implant comprising (a) providing a polymeric material; (b) blending the polymeric material with at least one therapeutic additive; and (c) consolidating the blend; thereby obtaining a therapeutic, medical implant.

In one embodiment, the invention provides a method of making a therapeutic medical implant comprising (a) providing a polymeric material; (b) blending the polymeric material with at least one therapeutic additive; (c) consolidating the blend; and (d) machining the consolidated blend; thereby obtaining a therapeutic, medical implant.

In one embodiment, the invention provides a therapeutic medical implant made by a method comprising (a) providing a polymeric material; (b) blending the polymeric material with at least one therapeutic additive; and (c) consolidating the blend; thereby obtaining a therapeutic, medical implant.

In some embodiments, the invention provides a therapeutic medical implant made by any of the described embodiments.

In one embodiment, the invention provides a method of making an oxidation-resistant therapeutic medical implant comprising (a) providing a polymeric material; (b) blending the polymeric material with an antioxidant; (c) blending the antioxidant-blended polymeric material with at least one therapeutic additive; and (d) consolidating the blend; thereby obtaining an oxidation-resistant therapeutic medical implant.

In one embodiment, the invention provides a method of making an oxidation-resistant therapeutic medical implant comprising (a) providing a polymeric material; (b) blending the polymeric material with an antioxidant; (c) blending the antioxidant-blended polymeric material with at least one therapeutic additive; (d) consolidating the blend; (e) machining the consolidated blend; thereby obtaining an oxidation-resistant therapeutic medical implant.

In one embodiment, the invention provides a method of making an analgesic medical implant comprising (a) blending the polymeric material with lidocaine, bupivacaine, and/or ropivacaine; (b) consolidating the blend; and (c) machining the consolidated blend; thereby obtaining an analgesic medical implant.

In one embodiment, the invention provides an analgesic medical implant made by a method comprising (a) blending the polymeric material with lidocaine, bupivacaine, and/or ropivacaine; (b) consolidating the blend; and (c) machining the consolidated blend; thereby obtaining an analgesic medical implant.

In one embodiment, the invention provides a method of making an oxidation-resistant therapeutic medical implant comprising (a) providing a polymeric material; (b) blending the polymeric material with an antioxidant; (c) blending the antioxidant-blended polymeric material with bupivacaine; and (d) consolidating the blend; thereby obtaining an oxidation-resistant therapeutic medical implant.

In one embodiment, the invention provides a method of making an oxidation-resistant therapeutic medical implant comprising (a) providing a polymeric material; (b) blending the polymeric material with an antioxidant; (c) blending the antioxidant-blended polymeric material with bupivacaine; (d) consolidating the blend; and (e) machining the consolidated blend; thereby obtaining an oxidation-resistant therapeutic medical implant.

In any of the embodiments, the medical implant can be packaged and sterilized. In any of the embodiments, the medical implant can be exposed to irradiation for sterilization or for cross-linking or for both. Sterilization can be done by gas sterilization methods such as ethylene oxide or gas plasma sterilization or by radiation methods such as gamma, electron beam or ultraviolet or blue light irradiation.

### Blending in Layers/Sections and Consolidation

In one embodiment, the invention provides a method of making a therapeutic, layered polymeric material comprising (a) providing a polymeric material; (b) blending the polymeric material with one therapeutic additive; (c) providing a second polymeric material; (d) blending the second polymeric material with a second therapeutic additive; (e) layering the two blends of polymeric material; and (f) consolidating the layered blends; thereby obtaining a therapeutic layered polymeric material. The first and the second polymeric materials can be the same. The first and the second therapeutic agents can be the same. The first and the second therapeutic agents can be different forms of the same therapeutic agent, for example bupivacaine hydrochloride and bupivacaine free base. The layering can be done in any manner that will allow the consolidated material to have spatially controlled regions of therapeutic additives. The layers can comprise entire surface or surfaces of the medical device or implant or just sections of surfaces of the medical device or implant.

In one embodiment, the invention provides a method of making a therapeutic, layered polymeric material comprising (a) providing a polymeric material; (b) blending the polymeric material with at least one therapeutic additive; (c) providing a second polymeric material; (d) layering the two blends of polymeric material; and (e) consolidating the layered blends; thereby obtaining a therapeutic layered polymeric material. The layering can be done in any manner that will allow the consolidated material to have spatially controlled regions of therapeutic additives. The layers can comprise entire surface or surfaces of the medical device or implant or just sections of surfaces of the medical device or implant. The second polymeric material can contain no additives, or an additive without therapeutic properties.

In one embodiment, the invention provides a method of making a therapeutic, layered hybrid material comprising (a) providing a polymeric material; (b) blending the polymeric material with at least one therapeutic additive; (c) providing a second polymeric material; (d) providing a metallic surface; (e) layering the two blends of polymeric material and the metallic surface; and (f) consolidating the layered blends onto the metallic surface; thereby obtaining a therapeutic layered and hybrid material. The layering can be done in any manner that will allow the consolidated material to have spatially controlled regions of therapeutic additives. The layers can comprise entire surface or surfaces of the medical device or implant or just sections of surfaces of the medical device or implant. The second polymeric material can contain no additives, or an additive without therapeutic properties. The consolidation with the metallic surface is to obtain a strong and interlocked interface between metallic and polymeric components. The metallic surface can be pre-treated by physical or chemical methods. The metallic surface can be porous.

In one embodiment, the invention provides a method of making a layered therapeutic polymeric material comprising (a) providing a polymeric material; (b) blending the polymeric material with bupivacaine hydrochloride; (c) providing a second polymeric material; (d) blending the polymeric material with bupivacaine free base; (e) layering the two blends; and (f) consolidating the layered blends; thereby obtaining a layered therapeutic polymeric material.

In one embodiment, the invention provides a method of making a layered analgesic polymeric material comprising (a) providing a polymeric material; (b) blending the polymeric material with bupivacaine hydrochloride; (c) providing a second polymeric material; (d) blending the polymeric material with bupivacaine free base; (e) layering the two blends; and (f) consolidating the layered blends; thereby obtaining a layered analgesic polymeric material.

In one embodiment, the invention provides a method of making an analgesic wear-resistant polymeric material comprising the steps of: (a) providing a polymeric material; (b) blending the polymeric material with at least one analgesic agent; and (c) consolidating the analgesic-blended polymeric material, thereby forming the analgesic wear-resistant polymeric material.

In any of the embodiments, the polymeric material can be blended with at least one antioxidant. One of the antioxidants can be vitamin E.

In one embodiment, the invention provides a method of making a wear-resistant layered analgesic polymeric material comprising (a) providing a polymeric material; (b) blending the polymeric material with vitamin E; (c) blending the vitamin E-blended polymeric material with bupivacaine hydrochloride; (d) providing a second polymeric material; (e) blending the second polymeric material with vitamin E; (f) blending the vitamin E-blended second polymeric material with bupivacaine free base; (g) layering the two blends; and (h) consolidating the layered blends; thereby forming the wear-resistant layered analgesic polymeric material.

In one embodiment, the invention provides a method of making a therapeutic, layered medical implant comprising (a) providing a polymeric material; (b) blending the polymeric material with one therapeutic additive; (c) providing a second polymeric material; (d) blending the second polymeric material with a second therapeutic additive; (e) layering the two blends of polymeric material; and (f) consolidating the layered blends.

In one embodiment, the invention provides a method of making a layered therapeutic medical implant comprising (a) providing a polymeric material; (b) blending the polymeric material with bupivacaine hydrochloride; (c) providing a second polymeric material; (d) blending the polymeric material with bupivacaine free base; (e) layering the two blends; and (f) consolidating the layered blends.

In one embodiment, the invention provides a method of making a layered analgesic medical implant comprising (a) providing a polymeric material; (b) blending the polymeric material with bupivacaine hydrochloride; (c) providing a second polymeric material; (d) blending the polymeric material with bupivacaine free base; (e) layering the two blends; and (f) consolidating the layered blends.

In one embodiment, the invention provides a method of making a layered analgesic medical implant comprising (a) providing a polymeric material; (b) blending the polymeric material with vitamin E; (c) blending the vitamin E-blended polymeric material with bupivacaine hydrochloride; (d) providing a second polymeric material; (e) blending the second polymeric material with vitamin E; (f) blending the vitamin E-blended second polymeric material with bupivacaine free base; (g) layering the two blends; and (h) consolidating the layered blends.

In one embodiment, the invention provides a medical implant made by a method comprising (a) providing a polymeric material; (b) blending the polymeric material with one therapeutic additive; (c) providing a second polymeric material; (d) layering the two blends of polymeric material; and (e) consolidating the layered blends. The second polymeric material can contain no additives, can contain additives, or can contain additives that are not therapeutic.

In any of the embodiments where layering of polymeric material with additives such as analgesics before consolidation resulted in a consolidated polymeric material with spatially controlled regions of additive(s), the consolidated forms can be machined. Alternatively, they could be made in the form of or in a form that is close to that of the final medical implant.

In some embodiments the implant or preform has the anesthetic and/or analgesic throughout the entire thickness of the implant or preform. In some embodiments the anesthetic and/or analgesic concentration is nearly uniform throughout the entire implant. In other embodiments the anesthetic and/or analgesic concentration varies from region to region. In some embodiments the anesthetic and/or analgesic is in higher concentrations on surfaces that are exposed to bodily fluids, while other surfaces where the implant may be in contact with other metallic or non-metallic components, the anesthetic concentration is lower. In some embodiments the anesthetic and/or analgesic concentration is substantially higher near the articulating surfaces of the implant so that with articulation the elution of the anesthetic is accelerated and more AT/analgesic is delivered to the joint.

In another embodiment the implant or preform has the anesthetic and/or analgesic throughout the entire thickness of the implant or preform with a gradient in the concentration of the anesthetic and/or analgesic. In another embodiment the polymer-anesthetic and/or analgesic blend is molded onto the surface of a polyethylene implant or preform to obtain a surface containing the anesthetic and/or analgesic. In another embodiment the anesthetic and/or analgesic is found on a thin surface layer where the anesthetic and/or analgesic surface layer is selectively located at regions of the implant or preform from where elution is desired.

In any of the embodiments, the consolidated polymeric material with spatially controlled regions of additive(s) made into a medical implant can be packaged and sterilized. The polymeric material or medical implant can be packaged in vacuum, in inert gas, in sensitizing gas, in a solution, in a solution containing additive(s), in a solution containing therapeutics, in a solution containing anesthetics and/or analgesics. Sterilization can be done by gas sterilization methods, or radiation methods such as gamma irradiation.

### Blending/Consolidation and Diffusion

In one embodiment, the invention provides a method of making a therapeutic polymeric material comprising (a) providing a polymeric material; (b) consolidating the polymeric material; and (c) incorporating at least one therapeutic additive in the consolidated polymeric material by diffusion; thereby forming the therapeutic polymeric material.

In one embodiment, the invention provides a method of making a therapeutic medical implant comprising (a) providing a polymeric material; (b) consolidating the polymeric material; and (c) incorporating at least one therapeutic additive in the consolidated polymeric material by diffusion; thereby forming the therapeutic medical implant. The polymeric material can be consolidated into solid form in the shape of the medical implant directly or it could be machined after consolidation or after diffusion. Diffusion can be done in one step of exposure to the therapeutic additive in pure form, in a solution, in a dispersion, in an emulsion, in a gas, in a foam, in a supercritical fluid, in contact with a solid, paste or gel. Diffusion can be described as a combination of several steps where there is exposure of the polymeric material to the therapeutic additive using one or more of these methods and other steps to control the concentration and distribution of the additive in the polymeric material. For example, diffusion can be done by doping by exposure to the therapeutic additive followed by annealing at an elevated temperature. During the annealing step, the polymeric material or the medical implant could be maintained in inert gas, vacuum, in supercritical fluid, in air or in a controlled environment of different gases.

In one embodiment, the invention provides a method of making an analgesic polymeric material comprising (a) providing a polymeric material; (b) consolidating the polymeric material; and (c) incorporating bupivacaine free base in the consolidated polymeric material by diffusion; thereby forming the analgesic polymeric material.

In one embodiment, the invention provides a method of making an analgesic medical implant comprising (a) providing a polymeric material; (b) consolidating the polymeric material; and (c) incorporating bupivacaine free base in the consolidated polymeric material by diffusion.

In one embodiment, the invention provides a method of making an analgesic polymeric material comprising (a) providing a polymeric material; (b) consolidating the polymeric material; and (c) incorporating lidocaine in the consolidated polymeric material by diffusion.

In one embodiment, the invention provides a method of making an analgesic medical implant comprising (a) providing a polymeric material; (b) consolidating the polymeric material; and (c) incorporating lidocaine in the consolidated polymeric material by diffusion.

In one embodiment, the invention provides a method of making a therapeutic polymeric material comprising (a) providing a polymeric material; (b) blending the polymeric material with bupivacaine hydrochloride; (c) consolidating the polymeric blend; and (d) incorporating bupivacaine free base in the consolidated polymeric blend by diffusion.

In one embodiment, the invention provides a method of making a therapeutic medical implant comprising (a) providing a polymeric material; (b) blending the polymeric material with bupivacaine hydrochloride; (c) consolidating the polymeric blend; and (d) incorporating bupivacaine free base in the consolidated polymeric blend by diffusion.

In one embodiment, the invention provides a method of making a sterile, layered analgesic polymeric material comprising (a) providing a polymeric material; (b) blending the polymeric material with bupivacaine hydrochloride; (c) providing a second polymeric material; (d) blending the polymeric material with bupivacaine free base; (e) layering the two blends; (f) consolidating the layered blends; and (g) sterilizing the consolidated layered blend; thereby forming the sterile, layered analgesic polymeric material.

In one embodiment, the invention provides a method of making a sterile, layered medical implant comprising (a) providing a polymeric material; (b) blending the polymeric material with bupivacaine hydrochloride; (c) providing a second polymeric material; (d) blending the polymeric material with bupivacaine free base; (e) layering the two blends; (f) consolidating the layered blends; and (g) sterilizing the consolidated layered blend.

In one embodiment, the invention provides a method of making an irradiated, layered analgesic polymeric material comprising (a) providing a polymeric material; (b) blending the polymeric material with bupivacaine hydrochloride; (c) providing a second polymeric material; (d) blending the polymeric material with bupivacaine free base; (e) layering the two blends; (f) consolidating the layered blends; and (g) irradiating the consolidated layered blend; thereby forming the irradiated, layered analgesic polymeric material.

In one embodiment, the invention provides a method of making an irradiated, layered analgesic medical implant comprising (a) providing a polymeric material; (b) blending the polymeric material with bupivacaine hydrochloride; (c) providing a second polymeric material; (d) blending the polymeric material with bupivacaine free base; (e) layering the two blends; (f) consolidating the layered blends; and (g) irradiating the consolidated layered blend.

In one embodiment, the invention provides a method of making a sterile, therapeutic, consolidated polymeric material comprising (a) providing a polymeric material; (b) blending the polymeric material with at least one therapeutic additive; (c) consolidating the blend; and (d) sterilizing the consolidated blend; thereby forming the sterile, therapeutic, consolidated polymeric material.

In one embodiment, the invention provides a method of making a sterile, therapeutic medical implant comprising (a) providing a polymeric material; (b) blending the polymeric material with at least one therapeutic additive; (c) consolidating the blend; and (d) sterilizing the consolidated blend.

Yet in another embodiment the polymeric material is prepared in an implant shape (through machining and/or direct compression molding) and then soaked in the anesthetic and/or analgesic. In another embodiment the polymeric material is prepared in a preform shape, diffused with the anesthetic and/or analgesic, and machined into an implant shape.

In another embodiment the polyethylene implant is soaked in the anesthetic and/or analgesic in the operating room prior to implantation in the patient. In some embodiments the AT diffusion is carried out under an inert gas blanket to minimize the oxidation of the AT. In another embodiment the anesthetic and/or analgesic doping is done in a solution of the anesthetic and/or analgesic at various temperatures.

In any of the embodiments, a consolidated polymeric material can be machined at any time after consolidation. In any of the embodiments, a consolidated polymeric material can be machined into the near or exact shape of an implant. In any of the embodiments, any polymeric material prepared in the shape of an implant can be packaged and sterilized. In any of the embodiments, any polymeric material prepared in the shape of an implant can be irradiated. The irradiation dose can be at a dose sufficient to make the implant sterile or it could be at a dose higher than that sufficient to make the implant sterile.

In another embodiment polyethylene implant with the anesthetic and/or analgesic additive is packaged and sterilized using ionizing radiation, such as gamma, beta (e-beam), or x-ray irradiation. In some embodiments the implant is packaged in inert gas, such as nitrogen or argon, and sterilized. In some embodiments the implant is packaged in a vacuum package and sterilized. In some embodiments the implant is packaged in air and sterilized. In another embodiment polyethylene the implant with the anaesthetic and/or analgesic additive is packaged and sterilized using gas sterilization methods, such as ethylene oxide gas or gas plasma.

In another embodiment the implant with the anaesthetic and/or analgesic additive is packaged in the anaesthetic and/or analgesic or the anaesthetic and/or analgesic solution and sterilized using methods such as, ionizing radiation or gas sterilization.

In one embodiment, the invention provides a method of making a therapeutic cross-linked polymeric material comprising (a) providing a polymeric material; (b) consolidating the polymeric material; (c) cross-linking the polymeric material; and (d) incorporating at least one therapeutic additive in the consolidated cross-linked polymeric material by diffusion; thereby obtaining a therapeutic polymeric material. The cross-linking can be performed by any of the methods known to cross-link the polymeric material, for example by irradiation using ionizing radiation, ultraviolet radiation and chemical cross-linking. Cross-linking can result in a polymeric material with a spatial variation in cross-linking, for example the cross-linking can be higher in the surface(s) of the polymeric material. Chemical cross-linking can be achieved by incorporating, coating, doping any chemical which can cause cross-linking when triggered by the appropriate stimulus such as heating, cooling, radiation. Chemical cross-linking can be done by using organic peroxides as additives and stimulating their decomposition by heating. Chemical cross-linking can be done simultaneously or separate from consolidation.

In one embodiment, the invention provides a method of making a therapeutic cross-linked polymeric material comprising (a) providing a polymeric material; (b) consolidating the polymeric material; (c) cross-linking the polymeric material; (d) heating the polymeric material; and (e) incorporating at least one therapeutic additive in the consolidated cross-linked polymeric material by diffusion; thereby forming the therapeutic polymeric material.

In one embodiment, the invention provides a method of making a therapeutic cross-linked polymeric material comprising (a) providing a polymeric material; (b) consolidating the polymeric material; (c) cross-linking the polymeric material; (d) heating the polymeric material; (e) machining the polymeric material; and (f) incorporating at least one therapeutic additive in the consolidated cross-linked polymeric material by diffusion; thereby forming the therapeutic polymeric material.

In one embodiment, the invention provides a method of making a therapeutic cross-linked medical implant comprising (a) providing a polymeric material; (b) consolidating the polymeric material; (c) cross-linking the polymeric material; (d) heating the polymeric material; (e) incorporating at least one therapeutic additive in the consolidated cross-linked polymeric material by diffusion; and (f) machining the therapeutic polymeric material; thereby forming the therapeutic cross-linked medical implant.

### Additive Incorporation and Cross-Linking/Grafting

In one embodiment, the invention provides a method of making a cross-linked, therapeutic polymeric material comprising (a) providing a polymeric material; (b) blending the polymeric material with at least one therapeutic additive; (c) consolidating the blend; and (d) cross-linking the consolidated blend; thereby forming the cross-linked therapeutic polymeric material. Cross-linking can be done by ionizing irradiation, ultraviolet irradiation, chemical cross-linking. Cross-linking agents or additives that can graft onto the polymeric material can be used during irradiation and/or cross-linking.

In one embodiment, the invention provides a method of making a cross-linked, therapeutic medical implant comprising (a) providing a polymeric material; (b) blending the polymeric material with at least one therapeutic additive; (c) consolidating the blend; and (d) cross-linking the consolidated blend.

In one embodiment, the invention provides a cross-linked, therapeutic medical implant made by a method comprising (a) providing a polymeric material; (b) blending the polymeric material with at least one therapeutic additive; and (c) consolidating the blend; (d) cross-linking the consolidated blend.

In one embodiment, the invention provides a method of making a cross-linked, therapeutic, layered polymeric material comprising (a) providing a polymeric material; (b) blending the polymeric material with one therapeutic additive; (c) providing a second polymeric material; (d) blending the second polymeric material with a second therapeutic additive; (e) layering the two blends of polymeric material; (d) consolidating the layered blends; and (f) cross-linking the consolidated polymeric material. The first and the second polymeric materials can be the same. The first and the second therapeutic agents can be the same. The first and the second therapeutic agents can be different forms of the same therapeutic agent; for example bupivacaine hydrochloride and bupivacaine free base. The layering can be done in any manner that will allow the consolidated material to have spatially controlled regions of therapeutic additives. The layers can comprise entire surface or surfaces of the medical device or implant or just sections of surfaces of the medical device or implant. The polymeric material can be consolidated directly into the shape of a medical implant or a medical implant can be fashioned from the consolidated solid form by an additional step for example by machining. Cross-linking can be done before or after the medical implant shape is obtained.

In some embodiments, cross-linking can be performed by ultraviolet irradiation in the presence of an initiator such as benzophenone or 4-hydroxybezophenone. Cross-linking can be performed by irradiation in the presence of a chemical cross-linking agent such as an organic peroxide.

In some embodiments, during irradiation, some additives such as MPC or SLIPS can be grafted onto the polymeric material. Exposure to these additives can be done in a solution, a dispersion, in gas, in a foam, in supercritical fluid. Similar to when additives are diffused into the polymeric material, diffusion of additives for grafting can be performed using similar methods.

In one embodiment, the invention provides a method of making a grafted, therapeutic, layered polymeric material comprising (a) providing a polymeric material; (b) blending the polymeric material with one therapeutic additive; (c) providing a second polymeric material; (d) blending the second polymeric material with a second additive; (e) layering the two blends of polymeric material; (f) consolidating the layered blends; and (g) grafting at least one additive onto the polymeric material.

In one embodiment, the invention provides a method of making a grafted, therapeutic, layered polymeric material comprising (a) providing a polymeric material; (b) blending the polymeric material with one therapeutic additive; (c) providing a second polymeric material; (d) blending the second polymeric material with a second additive; (e) layering the two blends of polymeric material; (f) consolidating the layered blends; (g) incorporating an initiator in the consolidated polymeric material; (h) exposing the polymeric material to at least one additive for grafting; and (i) using an external stimulus to initiate grafting.

In one embodiment, the invention provides a method of making a grafted, therapeutic, layered medical implant comprising (a) providing a polymeric material; (b) blending the polymeric material with one therapeutic additive; (c) providing a second polymeric material; (d) blending the second polymeric material with a second additive; (e) layering the two blends of polymeric material; (f) consolidating the layered blends; and (g) grafting at least one additive onto the polymeric material.

In one embodiment, the invention provides a method of making a grafted, therapeutic, layered medical implant comprising (a) providing a polymeric material; (b) blending the polymeric material with one therapeutic additive; (c) providing a second polymeric material; (d) blending the second polymeric material with a second additive; (e) layering the two blends of polymeric material; (f) consolidating the layered blends; (g) incorporating an initiator in the consolidated polymeric material; (h) exposing the polymeric material to at least one additive for grafting; and (i) using an external stimulus to initiate grafting.

In some embodiments, at least one initiator for grafting can be incorporated by blending with the polymeric material at the same time as the therapeutic additive or as a separate processing step. In some embodiments, at least one crosslinking agent can be incorporated by blending with the polymeric material at the same time as the therapeutic additive or as a separate processing step.

In some embodiments, the external stimulus for grafting can be irradiation, heating/cooling, changes in the environment such as pH or ionic strength. Irradiation can be ionizing such as gamma or electron beam irradiation or ultraviolet or visible light irradiation. The environment in which the external stimulus is applied can be vacuum, air, inert gas, supercritical fluid, liquid, a mixture of gases, liquids, fluids or solid, paste or gel.

In one embodiment, the invention provides a consolidated analgesic polymeric material comprising (a) a first layer of polymeric material blended with at least one analgesic agent; and (b) a second layer of polymeric material blended with at least one analgesic agent.

In another embodiment, the invention provides an analgesic medical implant comprising layers of polymeric materials, wherein the first and second layers of the polymeric materials are blended with at least one analgesic agent and consolidated.

In another embodiment, the invention provides an analgesic medical implant comprising layers of cross-linked polymeric materials, wherein the first and second layers of the polymeric materials are blended with at least one analgesic agent and consolidated, and wherein the consolidated layers of the polymeric material is cross-linked by ionizing radiation or chemical cross-linking.

In another embodiment, the invention provides a consolidated and cross-linked analgesic polymeric material comprising (a) a first layer of polymeric material blended with at least one analgesic agent; and (b) a second layer of polymeric material blended with at least one analgesic agent; wherein the consolidated layers of the polymeric material is cross-linked by ionizing radiation or chemical cross-linking.

In another embodiment, the invention provides a wear-resistant analgesic polymeric material comprising a polymeric material blended with at least one analgesic agent.

### BRIEF DESCRIPTION OF THE DRAWINGS

FIG. 1. Schematic descriptions of examples of representative joint implants with surface(s) containing therapeutics, for example analgesics.

FIG. 2. Bupivacaine release from Bupi-PE 1, Bupi-PE 2, Bupi-PE 3.

FIG. 3. (a) Bacterial bioluminescence of rat dorsum receiving either polyethylene without additives (Control) or bupivacaine-eluting UHMWPE (Bupi-PE). Higher value of bioluminescence indicates higher amount of live bacteria. (b) Plot of total bioluminescence vs time post-surgery for control and Bupi-PE. Data are displayed as mean±SE. *p<0.05.

FIG. 4. (a) Top left plug, bupivacaine eluting-polyethylene. Top right plug, conventional polyethylene. Bottom images: plugs implanted into a rat knee, lateral transcondylar approach. (b) Representative pressure of the four limbs measured by Tekscan®. (c) Ratio of total pressure by non-surgical hindlimb to surgical hindlimb (Pᵣₐₜᵢₒ) of both control and rats receiving Bupi-PE (treated).

FIG. 5. Wear resistance of flat UHMWPE with benzophenone crosslinking (Flat BP X-Link) and Bupi PE with benzophenone crosslinking (Bupi PE X-Link).

FIG. 6. Wear resistance of flat UHMWPE with irradiation crosslinking and Bupi PE with irradiation crosslinking.

FIG. 7. Surface topography of Bupi PE and irradiated Bupi PE (Bupi PE X-Link) before and after wear testing.

FIG. 8. Wear resistance of flat UHMWPE and Bupi PE treated with SLIPS.

FIG. 9. Wear resistance of flat UHMWPE with MPC grafting and Bupi PE with MPC grafting (Bupi PE+MPC).

FIG. 10. Fluorescence microscopy of surface of Flat PE and Bupi-PE under no compression (0 MPa) and compression (8 MPa). Black indicates area with no fluorescent lubricants (polymer area), grey indicates area containing fluorescent lubricants.

FIG. 11. The FTIR absorbance spectra of lidocaine-doped CISM UHMWPE.

FIG. 12. Lidocaine index profiles of the 0.25 wt % (a) and 0.50 wt % (b) lidocaine blended cubes through 6 weeks of elution in 40° C. DI H₂O.

FIG. 13. The lidocaine index measured by FTIR as a function of depth from the surface towards the bulk of 1 cm cubes doped with lidocaine by immersing at 100° C. for 10, 40 and 90 minutes (a) and up to 640 minutes (>10 hours; b).

FIG. 14. Lidocaine index profiles of CISM cubes doped in lidocaine for 640 minutes and subsequently eluted in 40° C. (a) and 100° C. (b) DI H₂O for up to 56 days.

FIG. 15. Lidocaine concentration profiles as a function of depth of CISM doped with lidocaine at 120° C. for up to 48 hours (a) and a comparison of doping profiles for samples doped at 100° C. and 120° C. (b).

FIG. 16. Elution profiles of CISM cubes doped at 100° C. for 6 hrs (a), 12 hrs (b) and 48 hrs (c) before elution at 40° C. in DI water.

### DETAILED DESCRIPTION OF THE INVENTION

An “anesthetic (AT)” refers to an agent when administered to a living being reduces the sensation of pain. In this application, ‘anesthetic’ is used interchangibly with ‘analgesic’. Examples of such agents are lidocaine, bupivacaine, and others including all aminoester-type (eg. benzocaine, chloroprocaine, cocaine, cyclomethycaine, dimethocaine, piperocaine, propoxycaine, procaine, proparacaine, tetracaine) and/or aminoamide-type local anaesthetics (eg. articaine, bupivacaine, cinchocaine, etidocaine, levobupivacaine, lidocaine, mepivacaine, prilocaine, ropivacaine, trimecaine) and/or opioid narcotics (eg. morphine, codeine, thebaine, hydromorphone, hydrocodone, oxycodone, oxymorphone, desomorphine, nicomorphine, dipropanoylmorphine, benzylmorphine, ethylmorphine, buprenorphine, fentanyl, pethidine, methadone, dextropropoxyphene, and/or non-steroidal anti-inflammatory agents (eg. aspirin, diflunisal, salsalate, ibuprofen, naproxen, fenoprofen, ketoprofen, flurbiprofen, oxaprozin, loxoprofen, indomethacine, sulindac, etodolac, ketorolac, diciofenac, nabumetone, piroxicam, meloxicam, tenoxicam, droxicam, lomoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone) and/or paracetamol type agents (eg. tylenol, panadol) or mixtures thereof. In some embodiments the anaesthetic is a mixture of the anaesthetic in a solvent. The solvent could be water, saline, isopropanol, ethanol, propanol or other solvents. One skilled in the art can choose any solvent that will dissolve the anaesthetic or anaesthetic in the mixture that one decides to use in preparing the implant. Some of the anesthetic and/or analgesics are available in their hydrochloride form. For instance, lidocaine or bupivacaine may be available, respectively, in lidocane hydrochloride or bupivacaine hydrochloride forms.

“Polymeric material” refers to large molecules or macromolecules composed of many repeating subunits. “Polymeric material” includes polyolefins such as polyethylene or polypropylene. Polyethylene can include low density polyethylene(s), and/or linear low density polyethylene(s) and/or high density polyethylene(s) and/or ultrahigh molecular weight polyethylene(s) or mixtures thereof. For example, ultra-high molecular weight polyethylene (UHMWPE) refers to linear non-branched chains of ethylene having molecular weights in excess of about 500,000, preferably above about 1,000,000, and more preferably above about 2,000,000. Often the molecular weights can reach about 8,000,000 or more. Initial average molecular weight refers to the average molecular weight of the UHMWPE starting material, prior to any irradiation. See U.S. Pat. No. 5,879,400, PCT/US99/16070, filed on Jul. 16, 1999, and PCT/US97/02220, filed Feb. 11, 1997. The term “polyethylene article” or “polymeric article” or “polymer” generally refers to articles comprising any “polymeric material” disclosed herein.

The term “polymeric material” refers to polyethylene, for example, hydrogels, such as poly (vinyl alcohol), poly (acrylamide), poly (acrylic acid), poly(ethylene glycol). Polymeric material can be in the form of resin, flakes, powder, consolidated stock and can contain additives such as anti-oxidants. The “polymeric material” also can be a blend of one or more of different resin, flakes or powder containing different concentrations of additives such as antioxidants. The polymeric material also can be a consolidated stock of these blends. Polymeric material can contain an antioxidant. Methods to prepare antioxidant containing polymeric material have been described, for example, in (Muratoglu US B461225B2) the contents of which are included here in their entirety by reference.

The term “polymeric materials” or “polymer” also include hydrogels, such as poly (vinyl alcohol), poly (acrylamide), poly (acrylic acid), poly(ethylene glycol), blends thereof, or interpenetrating networks thereof, which can absorb water such that water constitutes at least 1 to 10,000% of their original weight, typically 100 wt % of their original weight or more or 99% or less of their weight after equilibration in water.

“Polymeric material” or “polymer” can be in the form of resin, flakes, powder, consolidated stock, implant, and can contain additives such as antioxidant(s) or therapeutic agents. The “polymeric material” or “polymer” also can be a blend of one or more of different resin, flakes or powder containing different concentrations of additive(s) such as antioxidants and/or therapeutic agents and/or a chemical crosslinking agents and/or anti-crosslinking agents and/or crosslinking enhancers. The blending of resin, flakes or powder can be achieved by the blending techniques known in the art. The “polymeric material” also can be a consolidated stock of these blends.

“Anti-crosslinking agent” is used to describe additives which can hinder cross-linking when added to be polymeric material. Some free radical scavengers can act as anti-crosslinking agents. Some other chemicals such as solvents can also act as anti-crosslinking agents. “Crosslinking enhancer” is used to describe additives which can enhance or increase crosslinking when added to the polymeric material. Some chemicals with unsaturated groups such as acetylene or some solvents can act as crosslinking enhancers.

“Polymeric materials” or “polymers” can also include structural subunits different from each other. Such polymers can be di- or tri- or multiple unit-copolymers, alternating copolymers, star copolymers, brush polymers, grafted copolymers or interpenetrating polymers. They can be essentially solvent-free during processing and use such as thermoplastics or can include a large amount of solvent such as hydrogels. Polymeric materials also include synthetic polymers, natural polymers, blends and mixtures thereof. Polymeric materials also include degradable and non-degradable polymers.

The products and processes of this invention also apply to various types of polymeric materials, for example, any polypropylene, any polyamide, any polyether ketone, or any polyolefin, including high-density-polyethylene, low-density-polyethylene, linear-low-density-polyethylene, ultra-high molecular weight polyethylene (UHMWPE), copolymers or mixtures thereof. The products and processes of this invention also apply to various types of hydrogels, for example, poly(vinyl alcohol), poly(ethylene glycol), poly(ethylene oxide), poly(acrylic acid), poly(methacrylic acid), poly(acrylamide), copolymers or mixtures thereof, or copolymers or mixtures of these with any polyolefin. Polymeric materials, as used herein, also applies to polyethylene of various forms, for example, resin, powder, flakes, particles, powder, or a mixture thereof, or a consolidated form derived from any of the above. Polymeric materials, as used herein, also applies to hydrogels of various forms, for example, film, extrudate, flakes, particles, powder, or a mixture thereof, or a consolidated form derived from any of the above.

The term “cross-linking” refers to a processes that result in the covalent bonding of the parts of a material, for example polymer chains in a polymeric material. In the case of UHMWPE, which is a semi-crystalline polymer, there is covalent bonding of the polymer chains of the polymeric material. For instance, the cross-link density of polyolefins, such as polyethylene can be measured by swelling a roughly 3×3×3 mm cube of polymeric material in xylene. The samples are weighed before swelling in xylene at 130° C. for 2 hours and they are weighed immediately after swelling in xylene. The amount of xylene uptake is determined gravimetrically, and then converted to volumetric uptake by dividing by the density of xylene; 0.75 g/cc. By assuming the density of polyethylene to be approximately 0.94 g/cc, the volumetric swell ratio of cross-linked UHMWPE is then determined. The cross-link density is calculated by using the swell ratio as described in Oral et al., Biomaterials 31: 7051-7060 (2010) and is reported in mol/m³. The term ‘cross-linked’ refers to the state of polymeric material that is cross-linked to any level.

A “crosslinking agent” is a compound which can cause cross-linking in polymeric materials. Most often, cross-linking of the polymer follows a trigger which initiates the cross-linking process. For example, crosslinking can be initiated by irradiation with or without the presence of a crosslinking agent. Or, crosslinking can be initiated by ‘chemical’ means using a crosslinking agent. In the case of peroxides as cross-linking agent(s), heating to a temperature where the peroxide decomposes into free radicals, which are then transferred onto the polymer and initiate recombination reactions causing cross-linking, is required. Methods of ‘chemical crosslinking’ or cross-linking using crosslinking agent(s) is described in WO/2013/151960A2, which is hereby incorporated by reference in its entirety. In other cases, other stimuli may be used to trigger the reaction such as the application of ultraviolet light, heat, pressure or vacuum, contact with a particular solvent, or irradiation or combinations thereof. In this invention, the cross-linking agents used are often those that are commercially available and may contain impurities. In some embodiments, the cross-linking agents may be 100% pure or less. In some embodiments, the cross-linking agents are 80%, 85%, 90%, 91%, 92%, 93%, 94%, 95%, 96%, 97%, 98%, or 99% pure.

Typically, a crosslinking agent is defined as a compound which can chemically attach to two or more points on the polymeric material, creating a linkage between the same or different polymer chains. Crosslinking agent is also a compound that can initiate the processes that lead to the crosslinking of the polymeric material and the compound may or may not itself chemically attach to the polymer. For instance, the cross-linking agent may have a free radical, which may abstract a hydrogen from the polymeric material, creating a free radical on the polymeric material; subsequently such free radicals on the polymeric material can react with each other to form a cross-linked without chemically attaching the cross-linking agent to the polymeric material. In some embodiments, the unreacted cross-linking agent and/or the byproducts of the cross-linking agent are partially or fully extracted from the polymeric material after cross-linking. This extraction, among other methods, can include solvent extraction, emulsified solvent extraction, heat, and/or vacuum.

The term “additive” refers to any material that can be added to a base polymeric material in less than 50 wt/wt %. This material can be an organic or inorganic material with a molecular weight less than that of the base polymer. An additive can impart different properties to the polymeric material, for example, it can be a therapeutic agent, a nucleating agent, a cross-linking agent, an anti-crosslinking agent or an antioxidant or combinations thereof. Concentrations can be from 0.001 wt % to 50 wt %, or from 0.01 wt % to 20 wt %, or from 0.1 wt % to 10 wt %, or 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20 wt %, or more.

The term “therapeutic agent” or “therapeutic additive” refers to a chemical substance or a mixture thereof capable of eliciting a healing reaction from the human body. A therapeutic agent can be referred to also as a “drug” in this application. The therapeutic agent can elicit a response that is beneficial for the human or animal. Examples of therapeutic agents are antibiotics, anti-inflammatory agents, anesthetic agents, anticoagulants, hormone analogs, contraceptives, vasodlilators, vasoconstrictors, or other molecules classified as drugs in the art. A therapeutic agent can sometimes have multiple functions such as, anesthetic, analgesic and/or antibiotic. An anesthetic includes, one or more classes of topical or local anesthetic, including, without limitation, esters, such as benzocaine, chioroprocaine, cocaine, cyclomethycaine, dimethocaine/larocaine, piperocaine, propoxycaine, procaine/novacaine, proparacaine, and tetracaine/amethocaine. Anesthetics and analgesics can work by different mechanisms such as blocking sodium channels and can include amides, such as articaine, bupivacaine, cinchocaine/dibucaine, etidocaine, levobupivacaine, lidocaine/lignocaine, mepivacaine, prilocaine, ropivacaine, and trimecaine, or molecules of the opioid family, such as morphine, codeine, heroin, hydromorphone, levorphanol, meperidine, methadone, oxycodone, propoxyphene, fentanyl, methadone, naloxone, buprenorphine, butorphanol, nalbuphine and pentazocine. Local anesthetic can also include combinations of the above from either amides or esters. An antibiotic includes, without limitation, aminoglycosides, cephalosporins, chioramphenicol, clindamycin, erythromycins, fluoroquinolones, macrolides, azolides, metronidazole, penicillins, tetracyclines, trimethoprim-sulfamethoxazole and vancomycin.

The term ‘irradiation’ refers to exposing a material to radiation, for example ionizing radiation such as a gamma, electron, X-ray or ultraviolet (UV) radiation. ‘Radiation cross-linking’ refers to a radiation process intended to cross-link a material as a result of irradiation, for example exposing UHMWPE to gamma irradiation to cross-link the material. It also refers to the cross-linking in the material that has resulted from a radiation process. The radiation dose used can be from 0.0001 kGy to 100000 kGy, or 0.1 kGy to 1000 kGy, or from 1 kGy to 300 kGy, or about 100 kGy, or about 150 kGy, or about 175 kGy, or about 200 kGy. The radiation dose rate can be from 0.001 kGy/min to 100000 kGy/min, or from 0.1 kGy/min to 100 kGy/min, or from 1 kGy/min to 50 kGy/min, or about 25 kGy/min, or about 10 kGy/min, or about 100 kGy/min. Irradiation can be done in air, in vacuum, or partial gas environments, for example mixtures of oxygen and nitrogen. It can also be done in inert gas or partial inert gas. It can also be done at ambient temperature, or below or above ambient temperature. It can be done at elevated temperatures above ambient temperature. Irradiation temperature can be from −100° C. to 1000° C. or from 0° C. to 500° C. or from 20° C. to 200° C. or from 25° C. to 150° C., or at about 25° C., or about 70° C., or about 100° C., or about 120° C., or about 125° C. Methods of “exposing to radiation” or “irradiation” are described, for example in U.S. Pat. No. 7,381,752 (Muratoglu), U.S. Pat. No. 7,858,671 (Muratoglu et al.) and U.S. Pat. No. 6,641,617 (Merrill et al.), which are incorporated here by reference in their entirety. Also, methods of irradiation and treatments after irradiation are described, for example in U.S. Pat. No. 7,431,874 (Muratoglu et al.), U.S. Pat. No. 6,852,772 (Muratoglu et al.), U.S. Pat. No. 8,420,000 (Muratoglu et al.), U.S. Pat. No. 8,461,225 (Muratoglu et al.) and U.S. Pat. No. 8,530,057 (Muratoglu et al.), which are incorporated here by reference in their entirety.

The penetration depth of radiation can be controlled by methods such as those described in U.S. Pat. Nos. 7,381,752; 7,205,339; 7,790,779 (Muratoglu); and WO 2013170005 A1/US 20150151866. Electron irradiation, in general, results in more limited dose penetration depth, but requires less time and, therefore, reduces the risk of extensive oxidation if the irradiation is carried out in air. In addition if the desired dose levels are high, for instance 20 MRad, the irradiation with gamma may take place over one day, leading to impractical production times. On the other hand, the dose rate of the electron beam can be adjusted by varying the irradiation parameters, such as conveyor speed, scan width, and/or beam power. With the appropriate parameters, a 20 MRad melt-irradiation can be completed in for instance less than 10 minutes. The penetration of the electron beam depends on the beam energy measured by million electron-volts (MeV). Most polymers exhibit a density of about 1 g/cm³, which leads to the penetration of about 1 cm with a beam energy of 2-3 MeV and about 4 cm with a beam energy of 10 MeV. If electron irradiation is preferred, the desired depth of penetration can be adjusted based on the beam energy. Accordingly, gamma irradiation or electron irradiation may be used based upon the depth of penetration preferred, time limitations and tolerable oxidation levels. In particular, differing electron energies will result in different depths of penetration of the electrons into the polymer. The practical electron energies range from about 0.1 MeV to 16 MeV giving approximate iso-dose penetration levels of 0.5 mm to 8 cm, respectively.

The term “blending” refers to mixing of different components, often liquid and solid or solid and solid to obtain a homogeneous mixture of said components. Blending generally refers to mixing of a polymeric material in its pre-consolidated form with an additive. If both constituents are solid, blending can be done by using other component(s) such as a liquid to mediate the mixing of the two components, after which the liquid is removed by evaporating. If the additive is liquid, for example α-tocopherol, then the polymeric material can be mixed with large quantities of the said liquid. This high concentration blend can be diluted down to desired concentrations with the addition of lower concentration blends or virgin polymeric material without the additive to obtain the desired concentration blend. This technique also results in improved uniformity of the distribution of the additive in the polymeric material. Methods of blending additives into polymeric material are described, for example in U.S. Pat. No. 7,431,874 (Muratoglu at al.), U.S. Pat. No. 9,168,683 (Muratoglu et al.) and WO02007024684A2 (Muratoglu et al.), which are incorporated by reference in their entirety.

The term ‘diffusion’ refers to the net movement of molecules from an area of high concentration to an area of low concentration. In these embodiments, it is defined to be interchangeably used with ‘doping by diffusion’. The term “doping” refers to a general process (see, for example, U.S. Pat. No. 7,431,874), that is introducing additive(s) to a material. Doping may also be done by diffusing an additive into the polymeric material by immersing the polymeric material by contacting the polymeric material with the additive in the solid state, or with a bath of the additive in the liquid state, or with a mixture of the additive in one or more solvents in solution, emulsion, suspension, slurry, aerosol form, or in a gas or in a supercritical fluid. The doping process by diffusion can involve contacting a polymeric material, medical implant or device with an additive, such as vancomycin, for about an hour up to several days, preferably for about one hour to 24 hours, more preferably for one hour to 16 hours. The doping time can be from a second to several weeks, or it can be 1 minute to 24 hours, or it can be 15 minutes to 24 hours in 15 minute intervals. The environment for the diffusion of the additive (bath, solution, emulsion, paste, slurry and the like) can be heated to room temperature or up to about 200° C. and the doping can be carried out at room temperature or up to about 200° C. For example, when doping a polymeric material by an antioxidant, the medium carrying the antioxidant can be heated to 100° C. and the doping is carried out at 100° C. Similarly, when doping a polymeric material with therapeutic agent(s), the medium carrying the therapeutic agent(s) can be cooled or heated. Or the doping can be carried out at 5, 10, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 230, 240, 250, 260, 270, 280, 290, 300, 320 or 340° C. If the additive is a peroxide, the doping temperature may be below the peroxide initiation temperature, at the peroxide initiation temperature or above the peroxide initiation temperature or parts of the doping process may be done at different temperatures. A polymeric material incorporated with an additive by diffusion in such a way is termed an “additive-diffused” polymeric material. If the additive is a therapeutic agent, a polymeric material incorporated with the additive is termed a “therapeutic agent-diffused” polymeric material. Diffusion of additives such as antioxidants by high temperature doping and homogenization methods are described in Muratoglu et al. (U.S. Pat. No. 7,431,874), which in incorporated by reference in its entirety. If the additive is an anesthetic and/or analgesic (AT), a polymeric material incorporated with the additive is termed an “anesthetic and/or analgesic (AT)-diffused” polymeric material. “Diffusing with the anaesthetic and/or analgesic” refers to placing the implant or the polymeric material in contact with the anaesthetic and/or analgesic to allow for diffusion of the anaesthetic and/or analgesic into the implant or the polymer. The diffusion can be done by soaking the implant or the polymer in the AT or AT solution or any other medium containing AT. In one embodiment the polyethylene implant is soaked in AT for 1, 10, 20, 30, 40, 50, 60, 70, 80, 90, 100, 600, 1,500, 10,000 minutes or longer to diffuse AT in polyethylene. The temperature of the AT during soaking is 20, 30, 40, 50, 60, 70, 80, 90, 100° C. or higher. In some embodiments the AT is in solution. In some embodiments the solvent used to dissolve AT is water, alcohol, or a mixture thereof. The diffusion is accelerated by increasing temperature and/or pressure, or supercritical CO₂ is used to increase the diffusion rate of AT. Diffusing AT into the polymer, soaking the polymer in AT, or doping the polymer with AT are interchangeably used throughout this application to mean that AT is incorporated into the polymer.

The term “antioxidant” refers to alpha- and delta-tocopherol; propyl, octyl, or dodecyl gallates; lactic, citric, ascorbic, tartaric acids, and organic acids, and their salts; orthophosphates, lycopene, tocopherol acetate are generally known form of antioxidants (see, for example, U.S. Pat. No. 7,431,874). Antioxidants are also referred as free radical scavengers, include: glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids, including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox family; Irganox® and Irganox® B families including Irganox® 1010, Irganox® 1076, Irganox® 1330; Irgafos® family including Irgafos® 168; phenolic compounds with different chain lengths, and different number of OH groups; enzymes with antioxidant properties such as superoxide dismutase, herbal or plant extracts with antioxidant properties such as St John's Wort, green tea extract, grape seed extract, rosemary, oregano extract, mixtures, derivatives, analogues or conjugated forms of these. Antioxidants/free radical scavengers can be primary antioxidants with reactive OH or NH groups such as hindered phenols or secondary aromatic amines, they can be secondary antioxidants such as organophosphorus compounds or thiosynergists, they can be multifunctional antioxidants, hydroxylamines, or carbon centered radical scavengers such as lactones or acrylated bis-phenols. The antioxidants can be selected individually or used in any combination. The term “antioxidant” refers to alpha- and delta-tocopherol; propyl, octyl, or dedocyl gallates; actic, citric, ascorbic, tartaric acids, and organic acids, and their salts; orthophosphates, tocopherol acetate. Vitamin E is a preferred antioxidant.

The term ‘consolidation’ refers generally to processes used to convert the polymeric material resin, particles, flakes, i.e. small pieces of polymeric material into a mechanically integral large-scale solid form, which can be further processed, by for example machining in obtaining articles of use such as medical implants. Methods such as injection molding, extrusion, compression molding, iso-static pressing (hot or cold), or other methods known in the art can be used. In the present invention, consolidation of layers of polymeric material having different additives is described.

Consolidation can be performed by “compression molding”. In some instances consolidation can be interchangeably used with compression molding. The molding process generally involves:

i. heating the polymeric material to be molded,

ii. pressurizing the polymeric material while heated,

iii. keeping at temperature and pressure, and

iv. cooling down and releasing pressure.

Heating of the polymeric material can be done at a desired rate. Temperature can be increased linearly with time or in a step-wise fashion or at any other rate. Alternatively, the polymeric material can be placed in a pre-heated environment. In some embodiments, the polymeric material is placed into a mold for consolidation and the process (steps i-iv) is started without pre-heating. The mold for the consolidation can be heated together or separately from the polymeric material to be molded. Steps (i) and (ii), i.e. heating and pressurizing before consolidation can be done in multiple steps and in any order. For example, polymeric material can be pressurized at room temperature to a set pressure level 1, after which it can be heated and pressurized to another pressure level 2, which still may be different from the pressure or pressure(s) in step (iii). Step (iii), where a high temperature and pressure are maintained is the ‘dwell period’ where a major part of the consolidation takes place. One temperature and pressure or several temperatures and pressures can be used during this time without releasing pressure at any point. For example, dwell temperatures in the range of 135 to 350° C. and dwell pressures in the range of 0.1 MPa to 100 MPa or up to 1000 MPa can be used. The dwell time can be from 1 minute to 24 hours, more preferably from 2 minutes to 1 hour, most preferably about 10 minutes. The temperature(s) at step (iii) are termed ‘dwell’ or ‘molding’ temperature(s). The pressure(s) used in step (iii) are termed ‘dwell’ or ‘molding’ pressure(s). The order of cooling and pressure release (step iv) can be used interchangeably. In some embodiments the cooling and pressure release may follow varying rates independent of each other. In some embodiments, consolidation of polymeric resin or blends of the resin with additive(s) are achieved by compression molding. The dwell temperature and dwell time for consolidation can be changed to control the amount of integration.

Compression molding can also follow “layering” of different polymeric material; in these instances it is termed “layered molding”. This refers to consolidating a polymeric material by compression molding one or more of its pre-molded and resin forms, which may be in the form of flakes, powder, pellets or the like or consolidated or pre-molded forms in layers. This may be done such that there can be distinct regions in the consolidated form containing different concentrations of additives such as antioxidant(s), therapeutic agent(s) and/or crosslinking agent(s). Layering can be done any method that deposits desired polymeric material in desired locations. These methods may include pouring, scooping, painting, brushing spraying. This deposition can be aided by materials, templates and such supporting equipment that do not become an eventual part of the consolidated polymeric material. Whenever a layered-molded polymeric material is described and is used in any of the embodiments, it can be fabricated by:
(a) layered molding of polymeric resin powder or blends of polymeric material containing a specific additive(s) where one or more layers contain said additive and one or more layers do not contain said additive(s);
(b) molding together of layers of polymeric material containing different or identical concentration of additives such as therapeutic agent(s), antioxidant(s) and/or crosslinking agent(s).

Layering and spatial control of additive concentrations and polymeric material morphology are described in WO2008092047A1 (Muratoglu et al.), which is incorporated by reference in its entirety.

One or more of the layers can be treated before or during molding by heating, or high temperature melting. Methods of high temperature melting are described in WO2010096771A2 (Oral et al.), which is incorporated by reference in its entirety.

The layer or layers to be molded can be heated in liquid(s), in water, in air, in inert gas, in supercritical fluid(s) or in any environment containing a mixture of gases, liquids or supercritical fluids before pressurization. The layer or layers can be pressurized individually at room temperature or at an elevated temperature below the melting point or above the melting point before being molded together. The temperature at which the layer or layers are pre-heated can be the same or different from the molding or dwell temperature(s). The temperature can be gradually increased from pre-heat to mold temperature with or without pressure. The pressure to which the layers are exposed before molding can be gradually increased or increased and maintained at the same level.

During consolidation, different regions of the mold can be heated to different temperatures. The temperature and pressure can be maintained during molding for 1 second up to 1000 hours or longer. During cool-down under pressure, the pressure can be maintained at the molding pressure or increased or decreased. The cooling rate can be 0.0001° C./minute to 120° C./minute or higher. The cooling rate can be different for different regions of the mold. After cooling down to about room temperature, the mold can be kept under pressure for 1 second to 1000 hours. Or the pressure can be released partially or completely at an elevated temperature.

In some embodiments, the consolidated polymeric material is fabricated through “direct compression molding” (DCM), which is compression molding using parallel plates or any plate/mold geometry which can directly result in an implant or implant preform. Preforms are generally oversized versions of implants, where some machining of the preform can give the final implant shape. In some embodiments certain features of the final implant shape may be machined after direct compression molding.

In some embodiments, the pre-molded polymeric material is subjected to high temperature melting and subsequently direct compression molded. The direct compression molded polymeric material may be in its final implant shape. In some embodiments certain features of the final implant shape may be machined after direct compression molding. In certain embodiments, the pre-molded polymeric material contains cross-linking agents. In some embodiments the pre-molded polymeric material is subjected to irradiation before the subsequent direct compression molding.

Compression molding can also be done such that the polymeric material is directly compression molded onto a second surface, for example a metal or a porous metal to result in an implant or implant preform. This type of molding results in a “hybrid material” or “hybrid interlocked material” or “hybrid interlocked polymeric material” or “hybrid interlocked medical implant preform” or “hybrid interlocked medical implant” or a ‘monoblock construct’. Molding can be conducted with a second piece, for example a metal or metallic surface that becomes an integral part of the consolidated polymeric article. For example, a combination of antioxidant-containing polyethylene resin, powder, or flake and virgin polyethylene resin, powder or flake is direct compression molded into a metallic acetabular cup or a tibial base plate. The porous tibial metal base plate is placed in the mold, antioxidant blended polymeric resin, powder, or flake is added on top. Prior to consolidation, the pores of the metal piece can be filled with a waxy or plaster substance through half the thickness to achieve polyethylene interlocking through the other unfilled half of the metallic piece. The pore filler is maintained through the irradiation and subsequent processing (for example peroxide diffusion) to prevent infusion of components in to the pores of the metal. In some embodiments, the article is machined after processing to shape an implant. In some embodiments, there is more than one metal piece integral to the polymeric article. The metal(s) may be porous only in part or non-porous. In another embodiment, one or some or all of the metal pieces integral to the polymeric article is a porous metal piece that allows bone in-growth when implanted into the human body. In one embodiment, the porous metal of the implant is sealed using a sealant to prevent or reduce the infusion of antioxidant/peroxide (in diffusion steps after consolidation) into the pores during the selective doping of the implant. Preferably, the sealant is water soluble. But other sealants are also used. The final cleaning step that the implant is subjected to also removes the sealant. Alternatively, an additional sealant removal step is used. Such sealants as water, saline, aqueous solutions of water soluble polymers such as poly-vinyl alcohol, water soluble waxes, plaster of Paris, or others are used. In addition, a photoresist like SU-8, or other, may be cured within the pores of the porous metal component. Following processing, the sealant may be removed via an acid etch or a plasma etch. In these embodiments, the polymeric material, which is molded directly onto a second surface to form the hybrid interlocked polymeric material, maybe a pre-molded polymeric material with or without additives and/or cross-linking agents. In such embodiments the pre-molded polymeric material may be subjected to high temperature melting and/or radiation cross-linking. “Consolidation of the polyethylene powder or the polyethylene blended with anaesthetic” refers to the shaping of the powder or the blend in a mold, followed by heating and pressurization, to consolidate the powder into a state where it can be either in its final implant shape or in a state where it can be further machined to obtain an implant shape.

In some embodiments, the implant is a monoblock construct, in that it is made out of a polymer load bearing surface with a metallic or ceramic backside where the two components are intimately mated together. The AT is present in the polymer and/or in the metallic or ceramic components. In some embodiments the metallic or ceramic component has porous surfaces to allow for bone in growth; the pores in these porous surfaces are in some embodiments filled with the AT. The fill of the AT in the pores of the porous metal or porous ceramic regions is between 1% and 100% that between 1% and 100% of the pore volume is filled by the AT. More preferably the fill is between 5% of the volume of the pores and 90% of the volume of the pores. In some embodiments the fill is about 50 to 70% of the volume of the pores. In one embodiment the metallic or the ceramic component is separate from the polymeric component; the two are connected to each other during surgery by the use of a locking mechanism between the two mating surfaces. In this embodiment the AT is in the pores of the metal or the ceramic component. The AT is also in the polymeric component.

In some embodiments containing AT the implant made by the polymer or the polymer and metal and/or ceramic construct is stored at a temperature below room temperature to minimize changes in AT concentration profile and/or to minimize elution of the AT out of the implant. In one embodiment the implant is packaged, sterilized, and stored in a cold room.

The term “medical device”, refers to an instrument, apparatus, implement, machine, implant or other similar and related article intended for use in the diagnosis, treatment, mitigation, attenuation, cure, management, or prevention of disease in humans or other animals. An “implantable device” is a medical device intended to be implanted in contact with the human or other animal for a period of time. “Implant” refers to an “implantable medical device” where a medical device, is placed into contact with human or animal skin or internal tissues for a prolonged period of time, for example at least 2 days or more, or at least 3 months or more or permanently. Implants can be made out of metals, ceramic, polymers or combinations thereof. They can also comprise fluids or living tissues in part or in whole. An “implant” can refer to several components together serving a combined function such as “total joint implant” or it can refer to a single solid form such as an “acetabular cup” as a part. The term ‘medical implant’ refers to a medical device made for the purpose of implantation in a living body, for example and animal or human body. The medical implants include but are not limited to acetabular liners, tibial inserts, glenoid components, patellar components, and other load-bearing, articular components used in total joint surgery. While medical implants can be load-bearing to some extent some bear more load than others. For instance a tibial insert bears more load than a man-hole cover implant used to cover screw holes in acetabular shells. The term “permanent device” refers to a device that is intended for implantation in the body for a period longer than several months. Permanent devices include medical implants or devices, for example, acetabular liner, shoulder glenoid, patellar component, finger joint component, ankle joint component, elbow joint component, wrist joint component, toe joint component, bipolar hip replacements, tibial knee insert, tibial knee inserts with reinforcing metallic and polyethylene posts, intervertebral discs, sutures, tendons, heart valves, stents, and vascular grafts. The term “medical implant” refers to a device intended for implantation in animals or humans for short or long term use. The medical implants, according to an aspect of the invention, comprises medical devices including acetabular liner, shoulder glenoid, patellar component, finger joint component, ankle joint component, elbow joint component, wrist joint component, toe joint component, bipolar hip replacements, tibial knee insert, tibial knee inserts with reinforcing metallic and polyethylene posts, intervertebral discs, sutures, tendons, heart valves, stents, and vascular grafts, fracture plates.

The term “preform” refers to an implant that is an intermediate solid form that can be used for processing before fashioning a final implant. A preform can be machined from larger solid forms such as bar stock or can be directly consolidated such as by compression molding. It can be an oversized version of the final implant in ‘near net’ form or it can be a shape unrelated to the final implant form. “Preform shape” refers to the shape of the polymeric material that is subsequently machined to obtain the final shape of the implant. In some embodiments a preform is used so that any dimensional changes or surface changes that may occur during the anaesthetic soaking is machined away in the subsequent step of reducing the preform shape to the final implant shape. Typically the preform shape will be close to the net implant shape. In some cases the preform may be much larger than the implant shape.

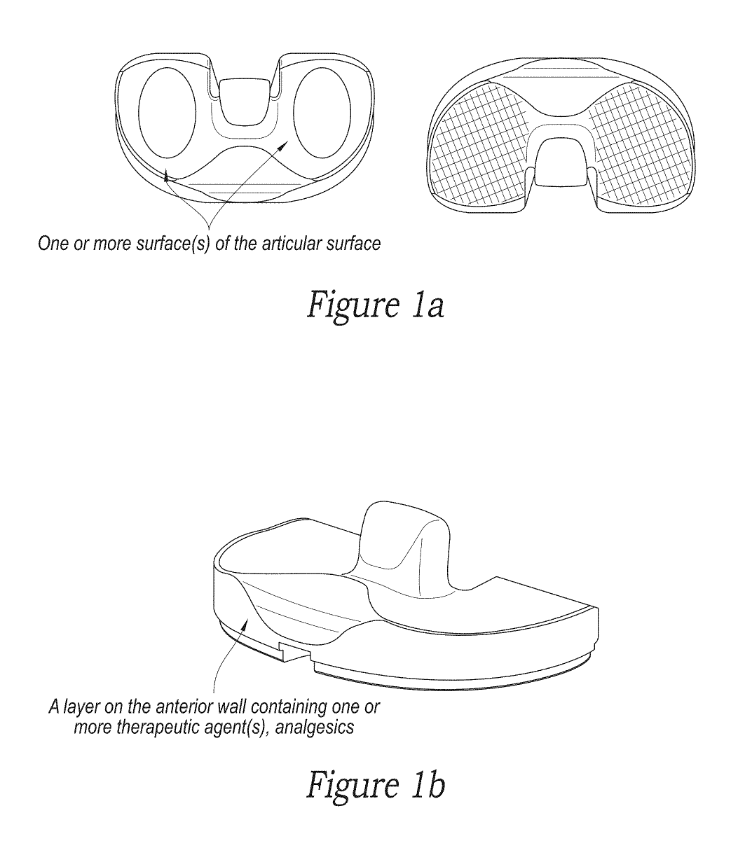

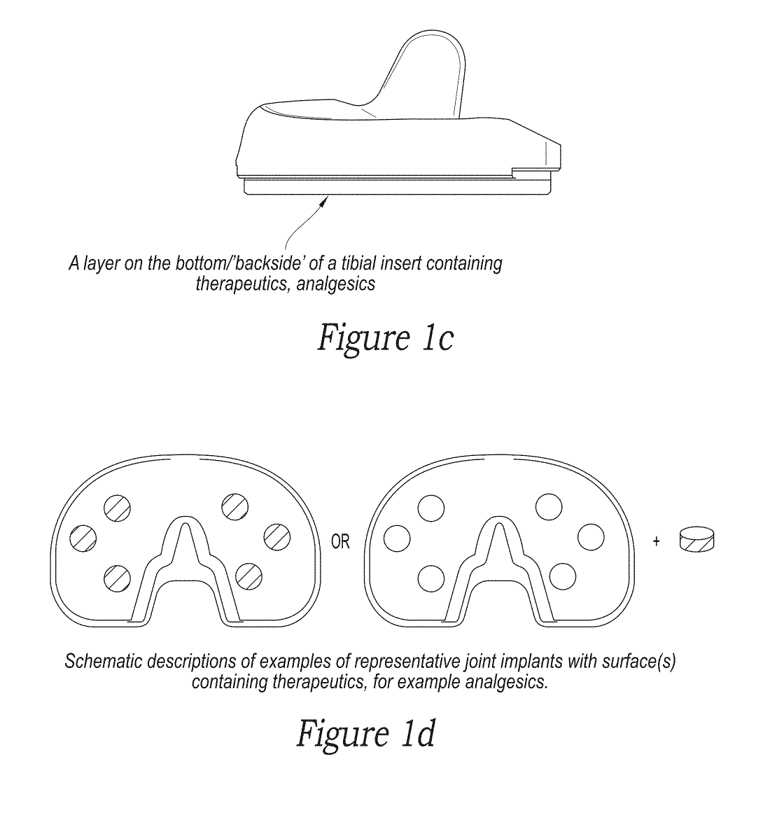

The term “surface” refers to any part of the outside of a solid-form material, which can be exposed to the surrounding liquid, gaseous, vacuum or supercritical medium. The surface can have a depth into the bulk of the material (normal to the surface planes), from several microns to several millimeters. For example, when a ‘surface layer’ is defined, the layer can have a thickness of several nanometers to several microns to several millimeters. For example, the surface layer can be 100 microns or 500 microns or 1000 microns (1 mm) or 2 mm or it can be between 2 and 5 mm. The surface or surfaces can also be defined along the surface planes. For example, a 5 mm wide and 15 mm long oval section of the articulating surface of a tibial knee insert can be defined as a ‘surface’ to be layered with a UHMWPE containing additives (FIG. 1a). These surfaces can be defined in any shape or size and the definition can be changed at different processing step. Some examples of surfaces are shown in FIG. 1. In some embodiments, an unloaded or minimally loaded region of the implant such as the anterior wall (FIG. 1b) or the backside (FIG. 1c) of the implant can contain therapeutic agents/analgesics. In some embodiments, the therapeutic polymeric material can be made separately from other part of a medical device and can fit on the surface(s) of the medical device before implantation. For example, in FIG. 1d, an example of a representative tibial knee insert is shown where there are regions on the backside (bottom) of the tibial insert containing an therapeutic agent/analgesic agent. These regions can be consolidated with the tibial insert or they can be prepared separately from the tibial insert and placed into pre-designed cutouts on the backside at the time of the surgery. These regions can be as thin as several hundred microns or up to several millimeters.

In some embodiments, the non-uniform distribution of the additive(s) within the implant is achieved by blending the additive with the polymeric material, such as polyethylene powder, and molding this polymeric material/additive blend along with virgin polyethylene (without additives) powder to obtain a spatially varying concentration of additive within the molded piece. In some embodiments, the additive can be a therapeutic agent. In some embodiments, the additive can be an anesthetic or analgesic agent. The methods by which such variations in additive concentration in an implant is achieved have been described for example by Muratoglu, Oral, and Kopesky in patent application US2010904481A, the contents of which are included here in their entirety by reference. In this application, the methods of spatially controlling the concentration of antioxidants are described.

The term “bupivacaine” refers to any form of the molecule which shows analgesic activity and relief of pain. This can be bupivacaine hydrochloride; its synonym is 1-Butyl-N-(2,6-dimethylphenyl)-2-piperidinecarboxamide hydrochloride. It is a solid at room temperature. It is white in color. Its melting point is 255° C. It is easily soluble in methanol, soluble in cold water, and partially soluble in diethyl ether. Alternatively, it can be bupivacaine free base. Its melting point is 120° C. It is highly hydrophobic and poorly soluble in water.

The term “lidocaine” refers to lidocaine, or lidocaine hydrochloride; its synonym is 2-(Dimethylamino)-N-(2,6-Dimethylphenyl)acetamide. It is a solid at room temperature. It is white to yellow in color. Its melting point is 68.5° C. Its boiling point is 181° C. It is easily soluble in diethyl ether and alcohol, but insoluble in cold water and hot water.

The term “bupivacaine index” refers to integrating the FTIR signal across 1627-1740 cm⁻¹, and normalizing it to the signal across 1850-1985 cm⁻¹.

The term “lidocaine index” refers to integrating the FTIR signal across 1627-1740 cm⁻¹, and normalizing it to the signal across 1850-1985 cm⁻¹.

The term “metallic material” refers to cobalt-chromium and its alloys with other metals, titanium and its alloys with other metals, and/or stainless steel and its alloys with other metals.

The terms “about” or “approximately” in the context of numerical values and ranges refers to values or ranges that approximate or are close to the recited values or ranges such that the invention can perform as intended, such as utilizing a method parameter (e.g., time, dose, dose rate/level, and temperature), having a desired degree of cross-linking and/or elution rate of an anesthetic and/or analgesic agents, as is apparent to the skilled person from the teachings contained herein. This is due, at least in part, to the varying properties of polymer compositions. Thus, these terms encompass values beyond those resulting from systematic error. These terms make explicit what is implicit, as known to the person skilled in the art.

### EXAMPLES

### Example 1. Manufacture of Bupivacaine Hydrochloride (Bupi-HCl) Eluting Polyethylene (Bupi-PE 1)

Bupivacaine hydrochloride (Bupi-HCl) (Sigma Aldrich, USA) was crushed and sieved through a 75 um sieve. Two grams of bupivacaine (HCl) powder was then mixed with 8 gram of GUR1050 UHMWPE powder until a homogeneous mixture was obtained. The resulting mixture was then transferred to the female part of a stainless steel mold (11 mm diameter). The male part of the mold was then placed in place and compression molded at 20 MPa between platens pre-heated to 170° C. for 5 minutes. The sample was then cooled under pressure to about room temperature at an approximate rate of 3° C./min. The pressure was then released to complete compression molding of bupivacaine-eluting polyethylene (Bupi-PE 1).

### Example 2. Manufacture of Bupivacaine Free Base Eluting Polyethylene Through Diffusion (Bupi-PE 2)

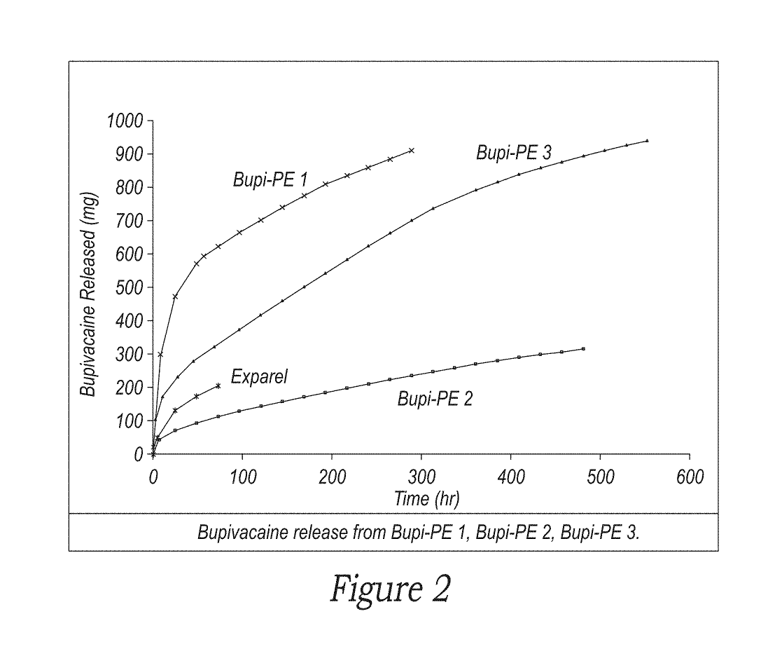

Consolidated polyethylene without any additives during consolidation was machined into 1 cm cubes, which were then immersed in pure bupivacaine free base at 150° C. under an argon purge for 10 minutes to 72 hr. In this way, a ‘bupivacaine-diffused’ UHMWPE was prepared. Bupi-PE 2 which has been prepared by doping for 48 hours is shown in FIG. 2.

### Example 3. Manufacture of a Layered Bupivacaine Hydrochloride Eluting Polyethylene on Virgin Polyethylene

Bupivacaine HCl (Sigma Aldrich, USA) was crushed and sieved through a 75 um sieve. Two grams of bupivacaine powder was then mixed with 8 grams of GUR1050 UHMWPE until a homogeneous mixture was obtained. The resulting mixture was then transferred to the female part of a stainless steel mold (11 cm diameter) and flattened. 90 grams of virgin polyethylene powder (without additives) was then layered on top of the bupivacaine HCL-blended powder and was also flattened. The male part of the mold was then placed and the layers were consolidated by compression molding at 20 MPa at 170° C. for 45 minutes and subsequent cooling under pressure to about room temperature at an approximate rate of 3° C./min.

### Example 4. Manufacture of a Layered Polyethylene Containing Bupivacaine Hydrochloride and Bupivacaine Free Base Polyethylene (Bupi-PE 3)

Bupivacaine HCl (Sigma Aldrich, USA) was crushed and sieved through a 75 um sieve. Two grams of bupivacaine powder was then mixed with 8 grams of GUR1050 UHMWPE until a homogeneous mixture was obtained. The resulting mixture was then transferred to the female part of a stainless steel mold (11 cm diameter) and flattened. 90 grams of virgin polyethylene powder (without additives) was then layered on top of the bupivacaine HCL-blended powder and was also flattened. The male part of the mold was then placed and the layers were consolidated by compression molding at 20 MPa at 170° C. for 45 minutes and subsequent cooling under pressure to about room temperature at an approximate rate of 3° C./min. The resulting consolidated polymer was machined into cubes (1 cm), which were then immersed in pure bupivacaine free base at 150° C. under an argon purge for 10 minutes to 72 hr.

### Example 5. Total Bupivacaine Release Rate from Bupi-PE 1, Bupi-PE 2, and Bupi-PE 3

Materials prepared as discussed in Examples 1, 4 and 6 were prepared as 3 mm×5 mm×20 mm blocks and each block was immersed in 1 ml phosphate buffered saline (PBS) at 37° C. for 6 hours. After 6 hours, the blocks were removed from the saline and immersed in a fresh 1 ml saline solution until 24 hours. After 24 hours, the sample was then transferred into a fresh 1 ml saline solution and the process was repeated every 24 hours until 3 weeks. Bupivacaine eluted into the saline solutions was measured using ultraviolet-visual (UV-Vis) spectroscopy. Elution results are displayed in FIG. 2. As a clinically relevant control, the elution from a liposomal depot formulation of bupivacaine (Exparel™, Pacira Pharmaceuticals) was also shown.

These results suggest that by manipulating the concentration and the layering of the two states of bupivacaine as well as using different methods of incorporation of the bupivacaine into UHMWPE, the release profiles of bupivacaine from drug-eluting UHMWPE can be modified.

### Example 6. In Vivo Murine Antibacterial Efficacy of Bupi-PE 3

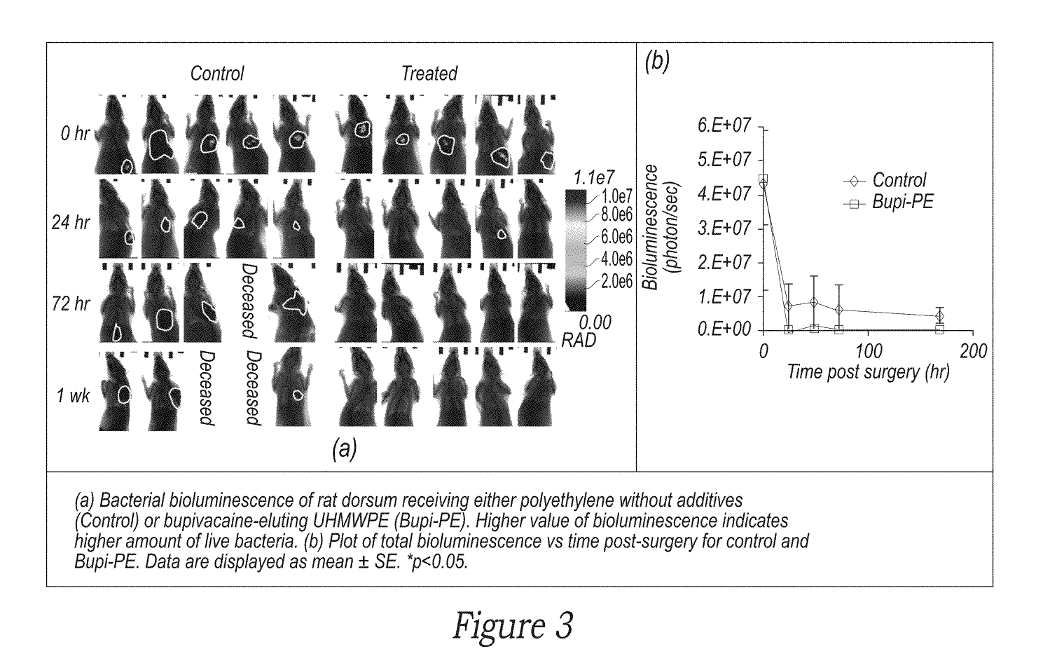

A total of n=10 male Sprague Dawley rats (250 g) were used in this study. Polyethylene without additives (control) or Bupi-PE 3 plugs (2.5 mm diameter×5 mm length) were implanted subcutaneously in the rat dorsum. After incision site closure, 5×10⁷ cfu of bioluminescent S. aureus (Xen 29) were injected around the implants. Bioluminescent signal (photos/second) was measured daily. All rats were euthanized after one week. One control rat expired at day 3 and another one expired at day 7. None of the rats receiving bupivacaine-eluting UHMWPE expired during the study. Significantly less bacterial load was observed in these rats, starting at 24 hr post implantation, continuing until the end of the study (day 7) (FIG. 3).

These results suggested that bupivacaine eluted from bupivacaine eluting UHMWPE (Bupi-PE 3) was able to eradicate S. Aureus in this lapine model of acute infection.

### Example 7. In Vivo Anesthetic Efficacy of Bupi-PE 3 in Murine Knee Joint Model

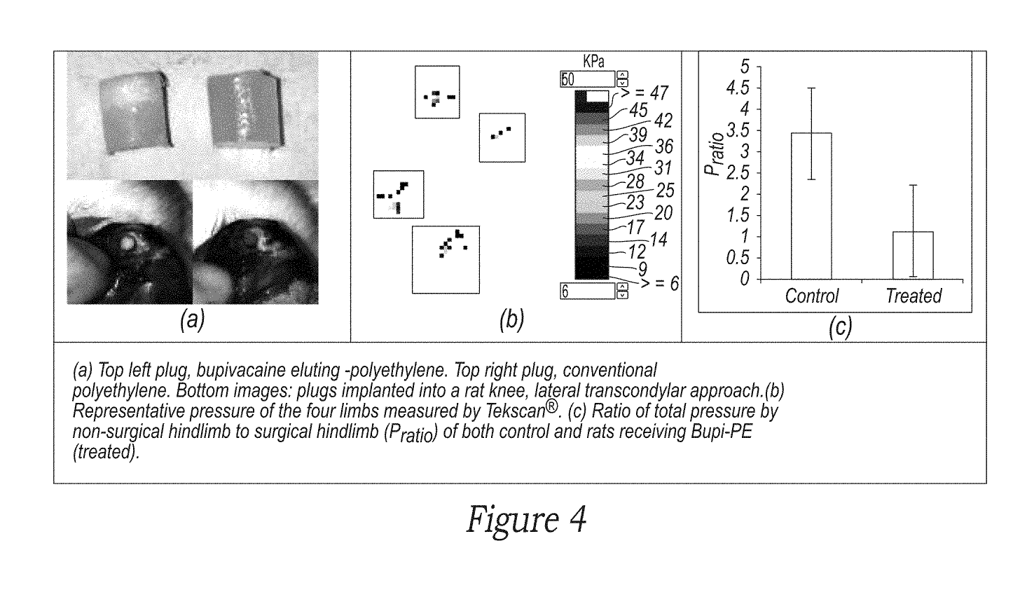

A total of n=10 male Sprague Dawley rats (250 g) were used in this study. Polyethylene without additives (control) and Bupi-PE 3 plugs (2.5 mm diameter×5 mm length) were implanted into rat knees via a lateral transcondylar approach (FIG. 4a). Analgesic efficacy of bupivacaine release was determined by performing a walking track analysis using a highly sensitive Tekscan® sensor (VHR, 5101) (FIG. 4b). Walking tracks were performed at baseline (pre-surgery) and every 24 hours for 2 weeks. All rats were euthanized after 2 weeks. Twenty four hours after surgery, rats in the control group loaded their unoperated hindlimb significantly more than their operated hindlimb. Rats with the bupivacaine-eluting UHMWPE implant loaded both their hindlimbs similarly (FIG. 4c).

These results suggested that bupivacaine release from bupivacaine-eluting UHMWPE had analgesic efficacy, resulting in the reduction of pain and the animals to be able to load their limbs in a normal fashion.

### Example 8: Benzophenone Crosslinked Bupi-PE

A Bupi-3 UHMWPE block prepared as described in Example 6 was washed twice with methanol in an ultrasonicator for 30 minutes twice. After the UHMWPE was dried, the block was immersed in a benzophenone solution in acetone (1.0 g/dl) for 30 seconds. The block was then dried in the absence of light for 1 hour in vacuum at room temperature. The benzophenone-coated UHMWPE block was placed in a glass vial filled with argon and then sealed. Photo-crosslinking on the polyethylene surface was carried out using an ultraviolet (UV) lamp (Dymax Bluewave 200, Torrington, Conn.) at 60° C. for 90 minutes. The resulting block was then washed with distilled water and hot ethanol (50° C.), and then dried in vacuum for 15 hours.

Wear testing of UHMWPE was adapted from Muratoglu et al. Biomaterials, 1999. 20(16): p. 1463-1470. Cylindrical pin shaped samples (9 mm in diameter and 11 mm in length) were used under bidirectional pin-on-disk testing at 2 Hz using a 10 mm×5 mm rectangular pattern. The loading cycle was adapted from Bergmann et al. J Biomech, 1993. 26(8): p. 969-990 and the human gait cycle was adapted from Muratoglu et al. Biomaterials, 1999. 20(16): p. 1463-1470. The UHMWPE pins were articulated against polished CoCr discs (Ra=0.38+/−0.005 um). Undiluted bovine serum was used as lubricant with 33 ml penicillin-streptomycin solution per 500 ml as antibacterial agent and 1 mM EDTA as chelating agent. The weight of the pin was measured every 0.1 million cycles. The wear rate values reported here are the cumulative weight values normalized by the total number of cycles and are averages of six pins.

The wear rate of bupi-PE cross-linked using benzophenone cross-linking showed much lower wear than that of benzophenone cross-linked UHMWPE without bupivacaine.

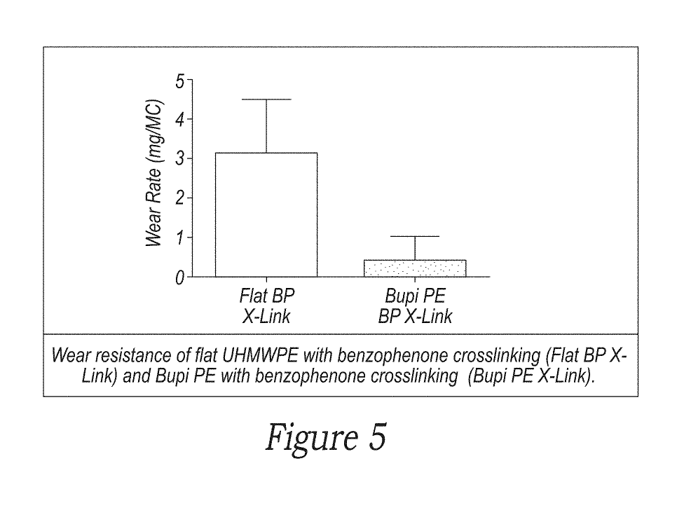

In another experiment, Bupi-PE 3 samples were packaged under vacuum in food packaging. Samples were then gamma-irradiated to 75 kGy. A control UHMWPE molded without bupivacaine (flat PE) was used. The wear rate of irradiated Bupi-PE was significantly lower than the wear rate of irradiated flat PE (FIG. 5).

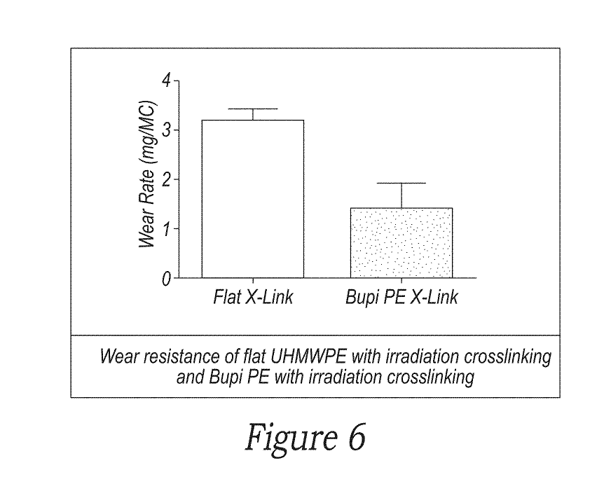

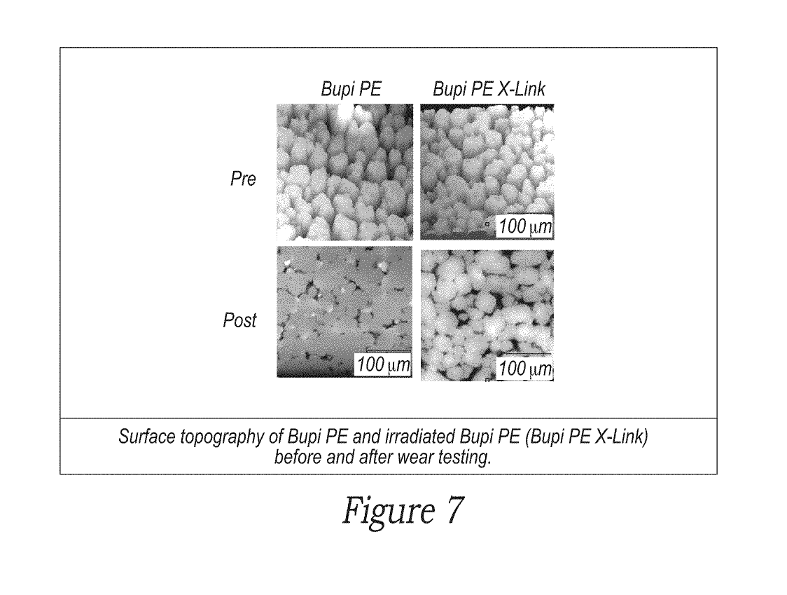

Topographical analysis of the micropatterns of Bupi-PE was performed and irradiated Bupi-PE before and after wear testing. Surface topology was analyzed using stylus profilometry (P16 Stylus Profiler, KLA Tencor) and atomic force microscopy (MFP-3D, Asylum). All stylus profilometry measurements were performed at room temperature in contact mode using scan speed of 10 um/s, sampling rate of 20 Hz, stylus radius of 0.5 um, and scan area of 1000 um×1000 um. There were no significant difference in the surface texture of the crosslinked and uncrosslinked Bupi-PE prior to wear testing (FIG. 6). However, after 1 million cycle of wear testing, the uncrosslinked Bupi-PE showed greater degree of flattening and smoothening than the crosslinked Bupi-PE (FIG. 7). These results suggested that cross-linking had reduced the deformation of the surface.

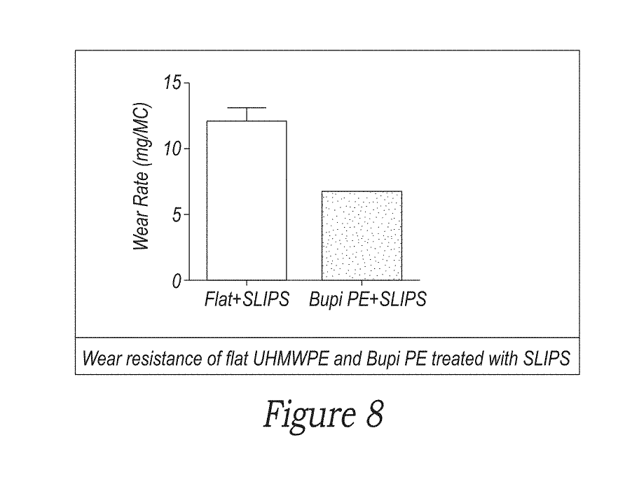

In another experiment, flat UHMWPE or Bupi-PE 3 was washed with methanol in ultrasonicator for 30 minutes and this procedure was repeated. After the UHMWPE was dried, the block was immersed in a benzophenone solution in acetone (1.0 g/dl) for 30 seconds. The block was then dried in the absence of light for 1 hr in a vacuum chamber at room temperature. A 2-methacryloyloxyethyl phosphorylcholine (MPC) solution (0.5 M) was prepared by dissolving 5 gram of MPC in 33.9 ml of degassed, double-distilled water. The benzophenone-coated UHMWPE block was placed in a glass vial filled with degassed MPC solution and sealed. Photopolymerization on the polyethylene surface was carried out using an ultraviolet (UV) lamp (Dymax Bluewave 200, Torrington, Conn.) at 60° C. for 90 minutes. The resulting block was then washed with distilled water and hot ethanol (50° C.), and then dried in vacuum for 15 hours. The wear rate of MPC grafted Bupi-PE is significantly lower than the wear rate of MPC grafted flat PE (FIG. 8).

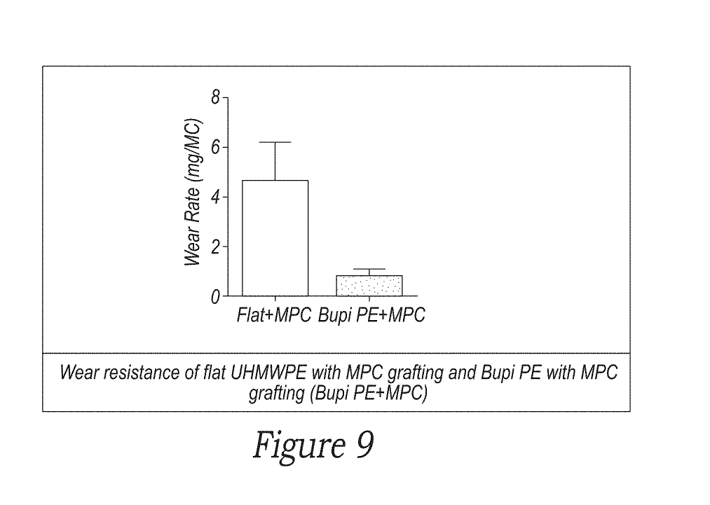

In another experiment, flat PE or Bupi PE block was exposed to 40 seconds of 250 mTorr radio-frequency (13.56 MHz) oxygen plasma at 100 Watts. The sample was then immersed in a liquid silane solution (5% v/v tridecafluoro-1,1,2,2-tetrahydrooctyl trichlorosilane) (Gelest, Morrisville, Pa.) in anhydrous ethanol (Sigma, St. Louis, Mo.)) for 1 hour at room temperature. It was then rinsed with anhydrous ethanol (Sigma, St. Louis Mo.), then with distilled DI water, and then three times with pure ethanol. It was then dried gently with nitrogen and then heated in an oven with desiccant at 60° C. for 12 hours at atmospheric pressure. The sample was then immersed in liquid perfluorodecalin (PFD) for 1 hour and then immediately tested. The wear rate of these slippery, liquid-infused in porous structure (SLIPS) treated Bupi-PE was significantly lower than the wear rate of SLIPS treated flat PE (FIG. 9).

### Example 9: Weeping Lubrication of Bupi-PE

Flat PE or Bupi PE was dipped in bovine serum with 0.1 wt % fluorescein dye (Sigma Aldrich, St. Louis, Mo.). The fluorescence was then measured from the surface of the samples under no compression pressure (0 MPa) and under compression (8 MPa).

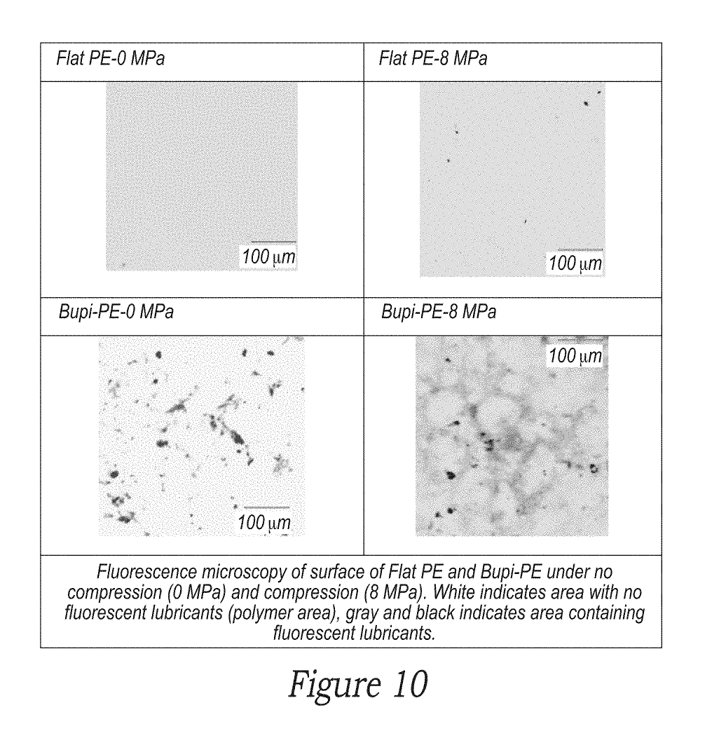

Dipping of Bupi PE in lubricant containing a fluorescent dye showed positive fluorescence in the valleys of the microtexture, indicating entrained lubricant (FIG. 10, 0 Mpa Bupi PE). Subsequent compression of the material showed an increased area of positive fluorescence, particularly originating from the valleys, indicating extrusion of lubricant from the compressed pores below the micro-textured surface (FIG. 10, 8 Mpa Bupi PE). On the other hand, dipping of the flat material in fluorescent dye with or without compression showed minimum fluorescence, indicating the absence of entrained lubricant.

### Example 10. The Determination of the Absorbance Signal and Integration Limits for Lidocaine and Bupivacaine by Fourier Transform Infrared Spectroscopy

A 100-kGy electron beam irradiation and melted UHMWPE (CISM) was machined into 1 cm cubes. A sliding microtome was used to cut 150 μm thin-sections. The resulting thin-sections were treated with a drop of a solution of isopropyl alcohol (IPA) containing lidocaine or bupivacaine. The following concentrations (wt/v %) were prepared: 0.00% (IPA-only Control), 0.50%, 1.00%, 1.25%, 2.50%, and 5.00%. The thin films were subsequently analyzed via FTIR, and the spectra of all samples were compared to determine the appropriate wave number (cm⁻¹) integration limits required to calculate a lidocaine index by which the concentration of lidocaine/bupivacaine in UHMWPE could be quantified.

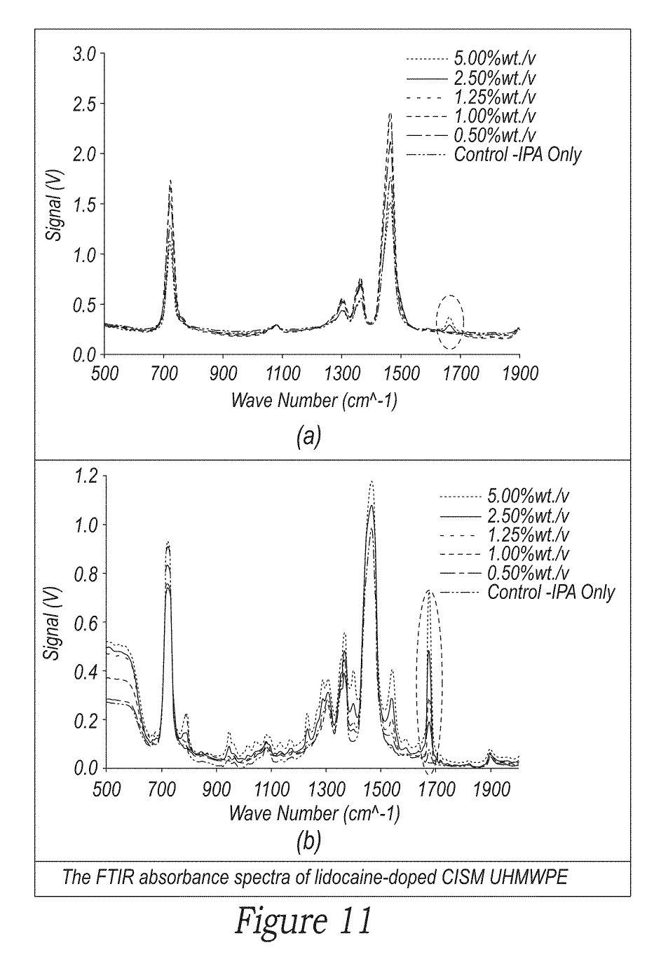

The FTIR spectra of CISM was notably affected by doping with the analgesic agents, with a peak signal occurring about 1674 cm⁻¹ (FIGS. 11a and 11b). Consequently, the lidocaine/bupivacaine index was calculated by integrating the FTIR signal across 1627-1740 cm⁻¹, and normalizing it to the signal across 1850-1985 cm⁻¹.

### Example 11. Manufacture of Lidocaine Eluting Polyethylene (Lido-PE) Through Blending

Lidocaine was dissolved in IPA, and the resulting solution was mixed with GUR1050 UHMWPE powder to yield blends containing 0.25 wt % and 0.50 wt % lidocaine. The lidocaine blends were then dried under vacuum for approximately 1 week to remove the solvent and subsequently consolidated by compression molding. The consolidated lidocaine blends were machined into 1 cm cubes. These cubes were then immersed in 40° C. DI water. Groups of n=3 cubes per each wt. % were removed from the DI water and analyzed via FTIR at the following time points: 0 weeks (Control), 1 week, 2 weeks, 4 weeks, and 6 weeks. FTIR spectra, and thereby lidocaine indices, were mapped across the width of each 1 cm cube.

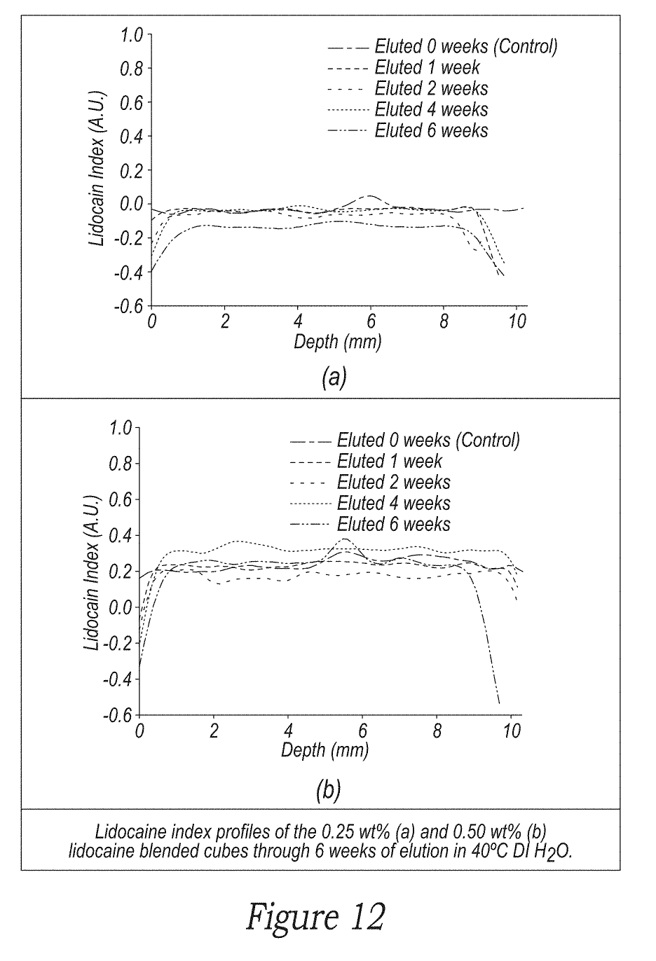

The splined average profiles of the three sections are shown in FIG. 12. The profiles showed a decrease in the surface concentration of lidocaine in UHMWPE with increasing elution time.

### Example 12. Manufacture of Low and High Dose Lidocaine Eluting Polyethylene Through Diffusion

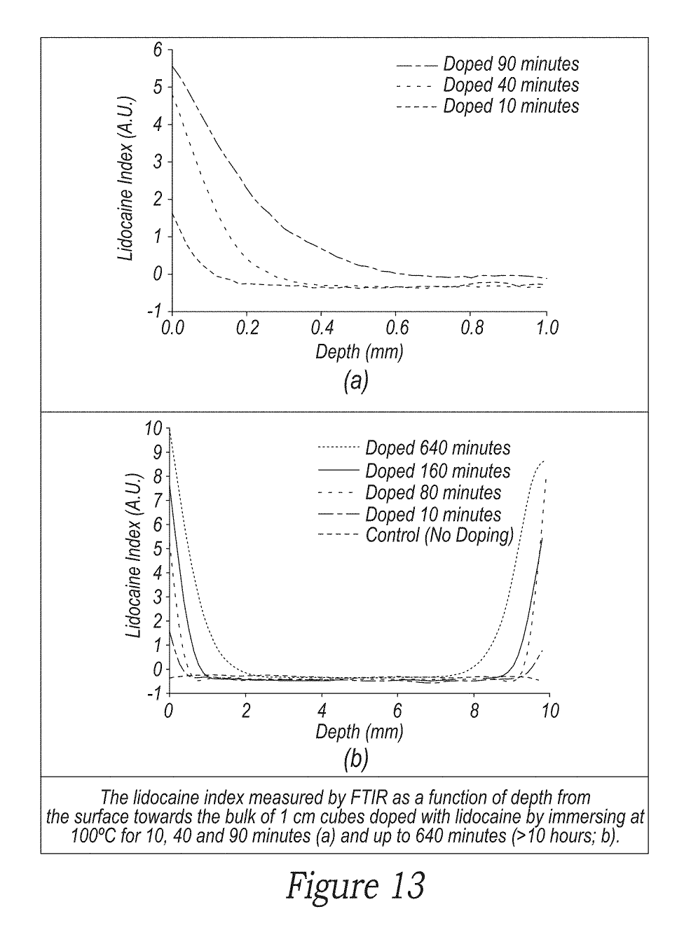

Consolidated polyethylene (without additives) subsequently irradiated to 100 kGy and melted was machined into 1 cm cubes, which were then immersed in pure lidocaine at 100° C. under an argon purge for 10 minutes, 40 minutes, 80 minutes, 90 minutes, 160 minutes, and 640 minutes. In this way, a ‘lidocaine-diffused’ UHMWPE was prepared. Fourier Transform infrared Spectroscopy was performed using a Varian 6701R/6201R FTIR Spectrometer. For all experiments involving 1 cm cubes, 150 μm thin-films were taken across the mid-height of each cube via a sliding microtome. These thin-sections were scanned such that FTIR spectra were mapped across the width of each 1 cm cube; data points were collected in 100 μm increments from the surface to a depth of 2 mm, and 500 μm increments throughout the bulk. The lidocaine indices were calculated by integrating the FTIR signal across 1627-1740 cm⁻¹, normalized to the signal across 1850-1985 cm⁻¹. The FTIR spectra of ‘lidocaine-diffused’ UHMWPEs prepared by immersion in lidocaine for different durations are shown in FIG. 13. Detectable lidocaine penetrated to a depth of 0.7 mm in the 90 minute doped group, 0.3 mm in the 40 minute doped group, and 0.2 mm in the 10 minute doped group, with maximum surface lidocaine indices of 5.5, 4.9, and 1.6, respectively (FIG. 13a).

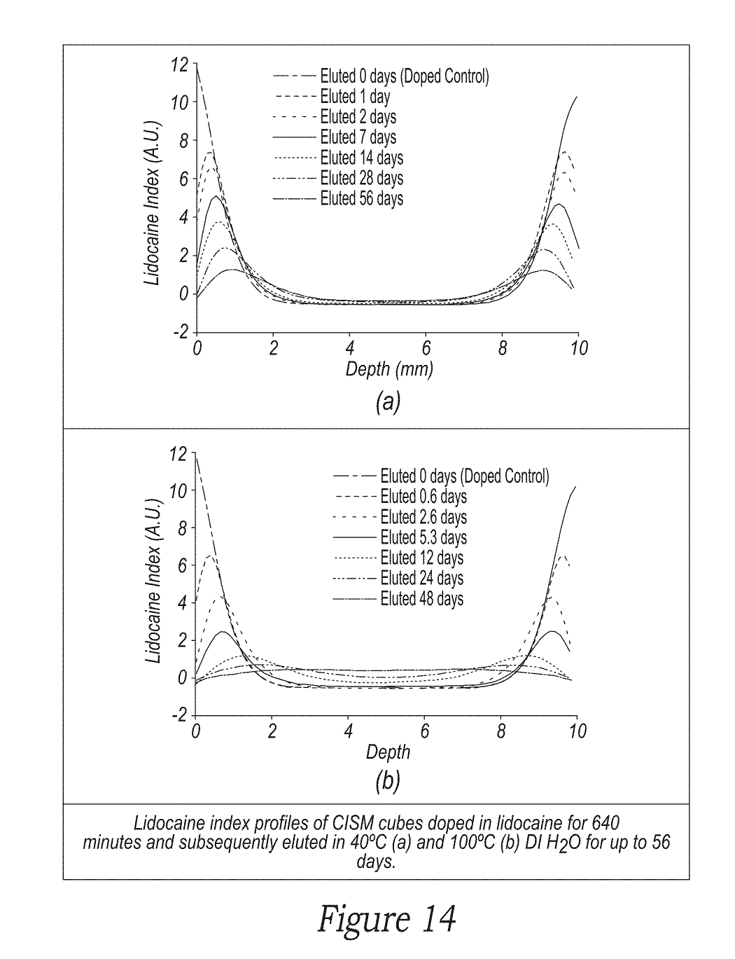

The ‘lidocaine-diffused’ UHMWPEs prepared by diffusion at 100° C. for 640 minutes were placed at 40° C. or 100° C. In deionized water for the subsequent elution of lidocaine. Three cubes each were eluted for 1, 2, 7, 14, 28 and 56 days. The cubes were microtomed perpendicular to a surface plane in the middle of the said surface and the FTIR spectra of the thin sections prepared as described above were obtained as a function of depth from the surface towards the bulk. The lidocaine profiles of eluted cubed are shown in FIG. 14.

The weight of lidocaine diffused into the cubes after doping for 640 minutes was 10.7±0.1 mg. The following amounts of lidocaine were eluted after the given time points in the 40° C. DI H₂O group: 1 day—1.4±0.1 mg (13%), 2 days—2.2±0.1 mg (20%), 7 days—3.6±0.1 mg (34%), 14 days—4.8±0.1 mg (45%), 28 days—6.3±0.2 mg (59%), and 56 days—7.5±0.1 mg (70%). In the 100° C. DI H₂O group, the following amounts of lidocaine were eluted after the given time points: 0.6 hours—2.0±0.0 mg (19%), 2.6 hours—4.1±0.1 mg (38%), 5.3 hours—5.2±0.0 mg (49%), 12 hours—7.3±0.0 mg (70%), 24 hours—8.6±0.2 mg (79%), 48 hours—9.0±0.1 mg (85%). FTIR analysis revealed that there was effectively no detectable lidocaine to be eluted after 48 hours of immersion in the 100° C. DI H₂O group (FIG. 14b), whereas there was still a marginal amount of detectable lidocaine remaining in the 40° C. DI H₂O group after 56 days (FIG. 14a)—a maximum lidocaine index of was still observed.

### Example 13. Pin-On-Disc (POD) Wear Testing of High Dose Lidocaine-Doped UHMWPE

Consolidated polyethylene (without additives) subsequently irradiated to 100 kGy and melted was machined into cylindrical pins (9 mm in diameter and 13 mm in height). One set of pins was immersed in 100° C. lidocaine for 640 minutes under an argon purge. Another untreated set of cylindrical pins was also machined. Prior to testing, both sets were immersed in 100° C. DI H₂O for 24 hours, removing a majority of the lidocaine absorbed during doping in the lidocaine-doped group. Each sample group consisted of n=3 cylindrical pins. A bi-directional POD wear tester was used to measure the wear rate of the UHMWPE samples articulated against polished Cobalt-Chrome (Co—Cr) discs, lubricated in preserved bovine serum. The bidirectional motion was produced by a computer-controlled XY table which was programmed to move in a 10 mm×5 mm rectangular pattern at 2 Hz. The MTS machine was programmed to produce a Paul-type load curve in synchronization with the motion of the XY table. The peak load corresponded to a peak contact pressure of 5.1 MPa between each UHMWPE pin and Co—Cr disc. The test was initially run for a bedding-in period of 0.5×10⁶ cycles, and the test was stopped at approximately every 0.157×10⁶ cycles for gravimetric assessment of wear until a total of 1.128×10⁶ cycles. The wear was determined by the gravimetric changes as a function of the number of cycles from 0.5 to 1.128 million cycles.

Lidocaine-doped CISM and untreated CISM, which were subsequently immersed in 100° C. DI H₂O for 24 hours, showed no significant difference in gravimetric loss during POD testing—with wear rates of −0.97±0.07 mg/Million Cycles, and −0.87±0.04 mg/Million Cycles, respectively (p-value=0.13).

### Example 14. High Dose Lidocaine Doping in Previously Cross-Linked UHMWPEs UHMWPEs

Consolidated polyethylene (without additives) was irradiated to 100 kGy and subsequently melted at about 170° (CISM) (see U.S. Pat. No. 8,076,387, for example). Consolidated polyethylene (without additives) was irradiated to 100 kGy, diffused by vitamin E, homogenized by heating, then terminally gamma sterilized (VEDI) (see U.S. Pat. No. 7,431,874, for example). Another type of UHMWPE was blended with 0.1 wt % vitamin E prior to consolidation, was mechanically deformed and thermally annealed to reduce the free radicals (eCIMA) (see U.S. Pat. No. 8,426,486, for example).

Machined (1 cm) cubes of VEDI, eCIMA, and CISM were immersed in 100° C. lidocaine under an argon purge for and 640 minutes. Each doping-time group contained n=6 cubes, which were weighed before and after lidocaine immersion to provide an assessment of the lidocaine absorbed.

After 640 minutes of doping in lidocaine, each material-group gained weight: VEDI gained 8.7±0.2 mg, eCIMA gained 10.5±0.3 mg, and CISM gained 11.0±0.1 mg. Each of these weight gains was significantly different from one another (p-value<0.005).

### Example 15. High Dose Lidocaine-Doped UHMWPE at 120° C.

Consolidated polyethylene (without additives) was irradiated to 100 kGy and subsequently melted at about 170° (CISM). CISM was machined into 1 cm cubes, which were then immersed in 120° C. lidocaine under an argon purge for 0 hours (Control), 0.16 hours, 1 hour, 2 hours, 6 hours, 12 hours, 24 hours and 48 hours. Each doping-time group contained n=3 cubes, which were weighed before and after lidocaine immersion to provide an assessment of the lidocaine absorbed. FTIR spectra, and thereby lidocaine indices, were mapped across the width of each 1 cm cube, as well.

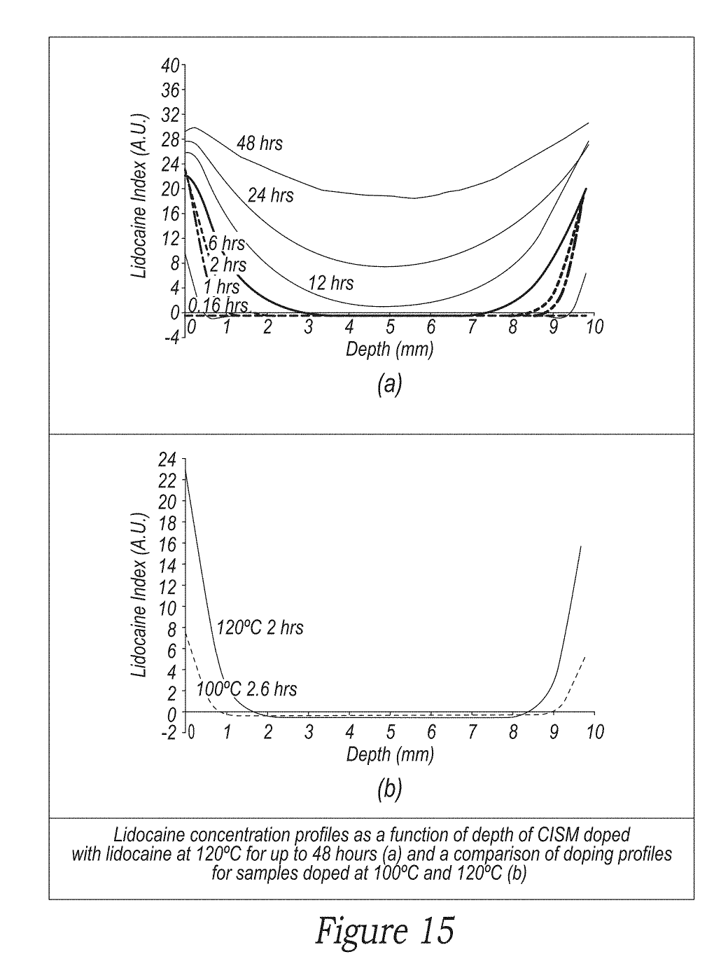

The following amounts of lidocaine were absorbed after the given doping times: 0.16 hours—3.0±0.1 mg, 1 hour—10.6±0.3 mg, 2 hours—16.8±0.1 mg, 6 hours—30.8±0.2 mg, 12 hours—40.3±0.2 mg, 24 hours—52.1±0.9 mg, 48 hours—61.9±0.4 mg. FTIR analysis revealed that the lidocaine index across the 48 hour group was nearly uniform, with the splined average of the lidocaine index residing between 30 and 20 A.U. (FIG. 15a). Furthermore, doping the CISM at 120° C. resulted in far greater lidocaine uptake than observed in doping at 100° C. (FIG. 15b). FTIR analysis revealed that the lidocaine index across the 48 hour group was nearly uniform.

CISM was machined into 1 cm cubes, which were then immersed in 120° C. lidocaine for 6 hours, 12 hours, and 48 hours. Afterwards, the cubes ware immersed in 40° C. DI H₂O. Groups of n=3 cubes were removed from the water for gravimetric assessment and FTIR analysis at the following time points: 0 hours (Control), 1 hour, 4 hours, 8 hours, 24 hours, 48 hours, 1 week, 2 weeks, and 4 weeks. FTIR spectra, and thereby lidocaine indices, were mapped across the width of each 1 cm cube in both groups.

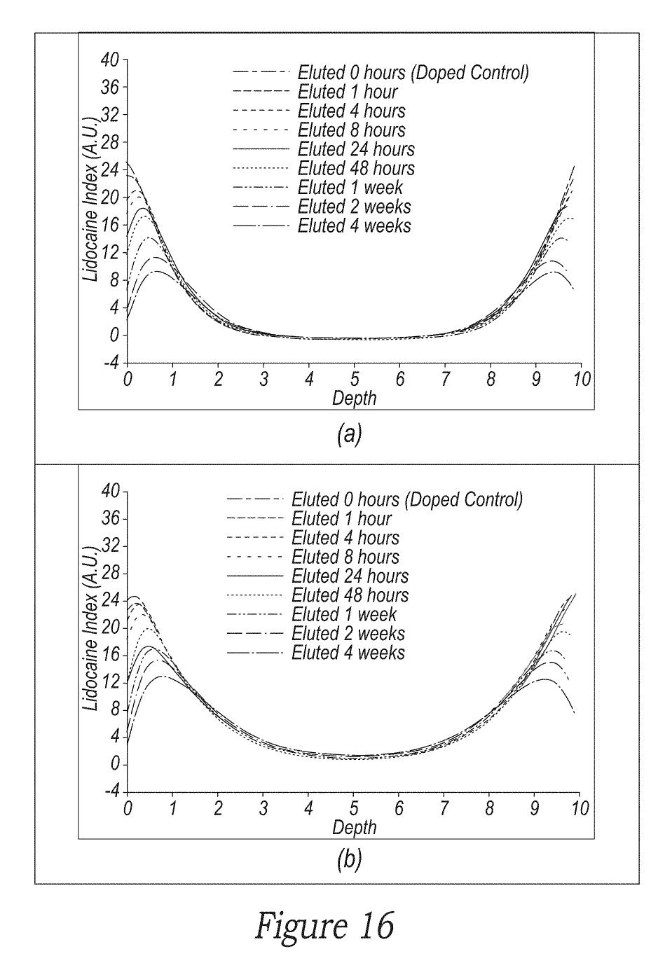

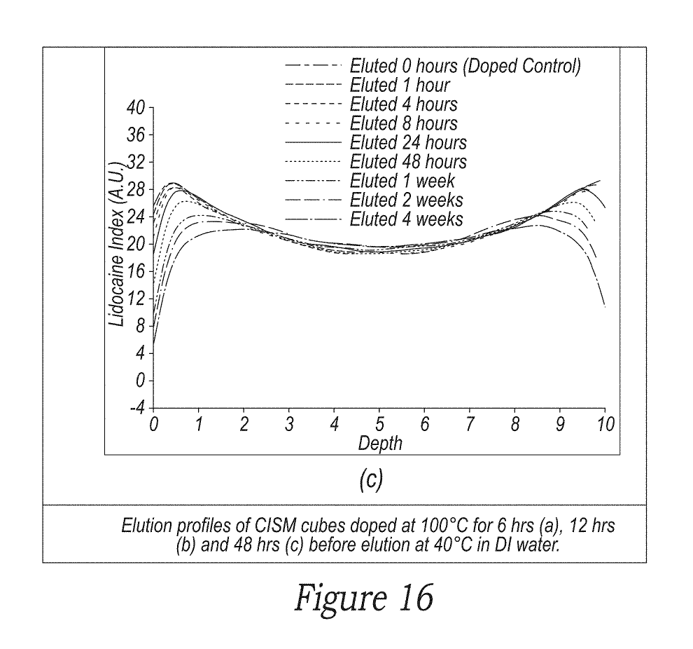

The greatest elution rate of lidocaine from each of the 1 cm cubes occurred between 0 and 1 hours, when the surface concentration of lidocaine was greatest Over the course of the first hour, the linearly approximated elution rate of each sample group was the following: 6 hour doped—0.39±0.04 mg/hour, 12 hour doped—0.37±0.04 mg/hour, and 48 hour doped—0.47±0.01 mg/hour. Elution rates decayed thereafter. After 8 hours, 1.49±0.08 mg had eluted from the 6 hour doped group, 1.54±0.03 mg had eluted from the 12 hour doped group, and 1.66±0.10 mg from the 48 hour doped group. After 4 weeks, 13.60±0.07 mg had eluted from the 6 hour doped group, 15.71±020 mg had eluted from the 12 hour doped group, and 18.82±0.10 mg from the 48 hour doped group. While the 48 hour doped cubes possess nearly twice as much lidocaine as the 6 hour doped group (approximately 62 mg vs. 31 mg), the elution rate did not scale proportionally. FTIR showed that lidocaine index profiles decreased commensurately (FIG. 16).

1-44. (canceled)

45. A method of making an analgesic polymeric material comprising the steps of:
a. providing a polymer;
b. blending the polymer with at least one analgesic agent; and
c. consolidating the polymer and at least one analgesic agent from step b, thereby forming the analgesic polymeric material.

46. The method of claim 45, further comprising blending the polymer with at least one selected form the group consisting of an antioxidant, an α-tocopherol, and vitamin E.

47. The method of claim 45, wherein at least one analgesic agent is selected from the group consisting of bupivacaine, ropivacaine, lidocaine, and ketorolac.

48. The method of claim 45, further comprising blending the polymer with at least one therapeutic agent.

49. The method of claim 48, wherein at least one therapeutic agent is an antibiotic selected from the group consisting of aminoglycosides, cephalosporins, chloramphenicol, clindamycin, erythromycins, fluoroquinolones, macrolides, azolides, metronidazole, penicillins, tetracyclines, trimethoprim-sulfamethoxazole, vancomycin and any combination thereof.

50. The method of claim 45, wherein the consolidated analgesic polymeric material is machined into a medical implant.

51. The method of claim 50, wherein the medical implant comprise at least one medical device selected from the group consisting of acetabular liner, shoulder glenoid, patellar component, finger joint component, ankle joint component, elbow joint component, wrist joint component, toe joint component, bipolar hip replacements, tibial knee insert, tibial knee inserts with reinforcing metallic and polyethylene posts, intervertebral discs, sutures, tendons, heart valves, stents, vascular grafts, and fracture plates.

52. The method of claim 45, wherein the consolidated analgesic polymeric material is cross-linked by ionizing radiation or chemical cross-linking, thereby forming a cross-linked analgesic polymeric material.

53. The method of claim 52, wherein the cross-linked analgesic polymeric material is machined to a final implant shape.

54. A method of making a layered, analgesic polymeric material comprising the steps of:
d. providing a first polymer;
e. blending the first polymer with at least one analgesic agent;
f. providing a second polymer;
g. layering the polymers from steps e and f; and
h. consolidating the layered polymers from step g, thereby forming the layered, analgesic polymeric material.

55. The method of claim 54, wherein the layered, analgesic polymeric material is sterilized, thereby forming a sterile, layered, analgesic polymeric material.

56. The method of claim 54, wherein at least the first polymer or second polymer are a blend with at least one selected from the group consisting of an antioxidant, an α-tocopherol, and vitamin E.

57. The method of claim 54, wherein at least one analgesic agent is selected from the group consisting of bupivacaine, ropivacaine, lidocaine, and ketorolac.

58. The method of claim 54, wherein the second polymer is blended with at least one analgesic agent.

59. The method of claim 54, further comprising blending at least the first polymer or the second polymer with at least one therapeutic agent.

60. The method of claim 59, wherein the therapeutic agent is an antibiotic selected from the group consisting of aminoglycosides, cephalosporins, chloramphenicol, clindamycin, erythromycins, fluoroquinolones, macrolides, azolides, metronidazole, penicillins, tetracyclines, trimethoprim-sulfamethoxazole, vancomycin and any combination thereof.

61. The method of claim 54, wherein is the layered, analgesic polymeric material is cross-linked by ionizing radiation or chemical cross-linking, thereby forming a cross-linked layered, analgesic polymeric material.

62. The method of claim 61, wherein the cross-linked layered, analgesic polymeric material is machined into a medical implant.

63. The method of claim 58, wherein the medical implant comprises at least one medical device selected from the group consisting of acetabular liner, shoulder glenoid, patellar component, finger joint component, ankle joint component, elbow joint component, wrist joint component, toe joint component, bipolar hip replacements, tibial knee insert, tibial knee inserts with reinforcing metallic and polyethylene posts, intervertebral discs, sutures, tendons, heart valves, stents, vascular grafts, and fracture plates.

64. The method of claim 45, wherein the analgesic polymeric material is machined into a first component that can be mated to at least a second component.

65. The method of claim 64, wherein the first component is connected to at least a second component.

66. The method of claim 65, wherein the first component is connected to at least a second component using a locking mechanism.

67. The method of claim 54, wherein the layered, analgesic polymeric material is machined into a first component that can be mated to at least a second component.

68. The method of claim 67, wherein the first component is connected to at least a second component.

69. The method of claim 68, wherein the first component is connected to at least a second component using a locking mechanism.

70. An analgesic polymeric material made according to the method of claim 45.

71. A medical implant comprising the analgesic polymeric material of claim 70.

72. A layered, analgesic polymeric material made according to the method of claim 54.

73. A medical implant comprising the layered, analgesic polymeric material of claim 72.
